(19) United States
(12) Patent Application Publication
(10) Pub. No.: US 2023/0391798 A1
(43) Pub. Date: Dec. 7, 2023

(54) DONOR-ACCEPTOR-DONOR TYPE MATERIALS FOR OPTOELECTRONIC APPLICATIONS

(71) Applicants: The University of Southern California, Los Angeles, CA (US); The Regents of the University of Michigan, Ann Arbor, MI (US)

(72) Inventors: Sunil K. Kandappa, Los Angeles, CA (US); Chandler Dobson, Los Angeles, CA (US); Mark E. Thompson, Los Angeles, CA (US); Peter I. Djurovich, Long Beach, CA (US); Yongxi Li, Ann Arbor, MI (US); Stephen R. Forrest, Ann Arbor, MI (US)

(21) Appl. No.: 18/322,819

(22) Filed: May 24, 2023

Related U.S. Application Data
(60) Provisional application No. 63/347,681, filed on Jun. 1, 2022.

(51) Int. Cl. C07D 519/00; C07F 5/02; H10K 30/50

(52) U.S. Cl. CPC C07D 519/00; C07F 5/022; H10K 30/50; H10K 2102/10

(57) ABSTRACT

Provided are compounds of Formula I. Also provided are formulations comprising these compounds. Further provided are optoelectronic devices that utilize these compounds.

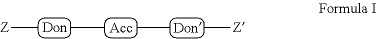

### CROSS-REFERENCE TO RELATED APPLICATIONS

The present application claims priority to U.S. Provisional Application No. 63/347,681, filed Jun. 1, 2022, which is incorporated by reference herein in its entirety.

### BACKGROUND

Optoelectronic devices rely on the optical and electronic properties of materials to either produce or detect electromagnetic radiation electronically or to generate electricity from ambient electromagnetic radiation. Opto-electronic devices that make use of organic materials are becoming increasingly desirable for a number of reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting diodes/devices (OLEDs), organic phototransistors, organic photovoltaic cells, and organic photodetectors.

Photosensitive optoelectronic devices convert electromagnetic radiation into electricity. Solar cells, also called photovoltaic (PV) devices or cells, are a type of photosensitive optoelectronic device that is specifically used to generate electrical power. PV devices, which may generate electrical energy from light sources other than sunlight, may be used to drive power consuming loads to provide, for example, lighting, heating, or to power electronic circuitry or devices such as calculators, radios, computers or remote monitoring or communications equipment. These power generation applications may involve the charging of batteries or other energy storage devices so that operation may continue when direct illumination from the sun or other light sources is not available, or to balance the power output of the PV device with the specific applications requirements.

Traditionally, photosensitive optoelectronic devices have been constructed of a number of inorganic semiconductors, e.g., crystalline, polycrystalline and amorphous silicon, gallium arsenide, cadmium telluride, and others.

More recent efforts have focused on the use of organic photovoltaic (OPV) cells to achieve acceptable photovoltaic conversion efficiencies with economical production costs. OPVs offer a low-cost, light-weight, and mechanically flexible route to solar energy conversion. Compared with polymers, small molecule OPVs share the advantage of using materials with well-defined molecular structures and weights. This leads to a reliable pathway for purification and the ability to deposit multiple layers using highly controlled thermal deposition without concern for dissolving, and thus damaging, previously deposited layers or subcells.

In addition to the pursuit of high device efficiency, OPVs have unique advantages, such as the application of semi-transparent solar cells for use in building integrated photovoltaics (BIPV). Considering the vast surface areas of windows and facades in modern urban environments, developing semi-transparent solar cells with both high efficiency and transmittance has become increasingly important. For a solar cell to be highly transparent, visible light would have to travel uninhibited to the eye, and hence cannot be absorbed. Selectively harvesting near-infrared (NIR) radiation avoids competition between efficiency and transmittance. However, the lack of high performance NIR absorbers in conventional fullerene based OPVs has prevented the attainment of efficient, yet highly transparent (in the visible) devices. To date, semi-transparent OPVs based on fullerene acceptors show only PCE less than or equal to 4% with average visible transmittance of 61%.

### SUMMARY OF THE INVENTION

In one aspect, the present invention relates to a compound of Formula I:

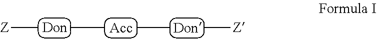

wherein Don and Don′ are each independently represented by Formula A or Formula B:

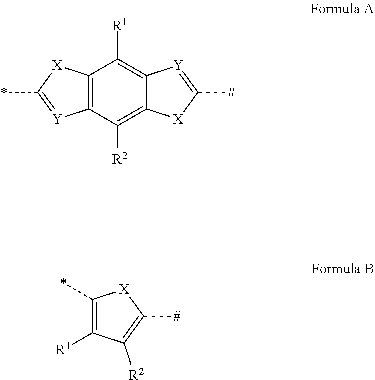

wherein, in Formula A and Formula B:
* represents the bond to Acc;
# represents the bond to Z or Z′;
each X is independently selected from the group consisting of O, S, Se, Te, GeRR′, CRR′, SiRR′, and NR.
each Y is independently selected from the group consisting of N and CR″.
R¹, R², R, R′, and R″ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and any two adjacent substituents optionally join to form a ring;
Acc is a divalent electron accepting group;
Z and Z′ are each together donating groups D and D′ respectively or accepting groups A and A′ respectively;
wherein each of Acc, Z, and Z′ may be further substituted with one or more substituents selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein any two adjacent substituents optionally join to form a ring.

In another aspect, the present disclosure provides a formulation comprising a compound of Formula I as described herein.

In yet another aspect, the present disclosure provides an optoelectronic device comprising a compound of Formula I as described herein.

### BRIEF DESCRIPTION OF THE DRAWINGS

FIG. 1 shows natural transition orbitals (NTOs) for BT-PCE-X-Y-structures.

FIG. 2 shows NTOs for further BT-PCE-X-Y structures.

FIG. 3 is a plot of normalized absorbance vs wavelength for BT-PCE and comparative compounds BT-CIC and Y6.

FIG. 4 is a depiction of the electrochemical band gap of inventive compound BT-PCE and comparative compounds BT-CIC and Y6.

FIG. 5 is plot of the absorption and emission spectra of BT-PCE.

FIG. 6 is a cyclic voltammogram of BT-PCE measured in DCM with supporting electrolyte (n-Bu)₄N⁺PF₆⁻.

FIG. 7 depicts the photovoltaic performance of BT-PCE.

FIG. 8 depicts the chemical structures of acceptor molecules chosen for DFT calculations and the corresponding HOMO LUMO values.

FIG. 9 is a table of calculated NTOs for BT-H-thiadiazole and BT-H-Py.

FIG. 10 depicts the UV-Vis absorption spectra of BT-thiadiazole.

FIG. 11 is a cyclic voltammogram of BT-thiadiazole measured in DCM with supporting electrolyte (n-Bu)₄N⁺PF₆⁻.

FIG. 12 shows calculated NTOs for azafulvene structures.

FIG. 13 shows additional NTOs for azafulvene structures.

FIG. 14 is plot of UV-Vis absorption of azafulvenes Az-H, Az-OMe, and AzOHex.

FIG. 15 is a cyclic voltammogram of Az-OMe measured in DCM with supporting electrolyte (n-Bu)₄N⁺PF₆⁻.

FIG. 16 is a cyclic voltammogram of Az-OHex measured in DCM with supporting electrolyte (n-Bu)₄N⁺PF₆⁻.

### DETAILED DESCRIPTION

The present disclosure relates in part to small molecules with an donor-acceptor-donor (DAD) type triad for organic photovoltaics (OPVs).

As used herein, the term “organic” includes polymeric materials as well as small molecule organic materials that may be used to fabricate organic opto-electronic devices. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety. The core moiety of a dendrimer may be a fluorescent or phosphorescent small molecule emitter. A dendrimer may be a “small molecule,” and it is believed that all dendrimers currently used in the field of optoelectronic devices are small molecules.

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processable” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

As used herein, the terms “electrode” and “contact” may refer to a layer that provides a medium for delivering current to an external circuit or providing a bias current or voltage to the device. For example, an electrode, or contact, may provide the interface between the active regions of an organic photosensitive optoelectronic device and a wire, lead, trace or other means for transporting the charge carriers to or from the external circuit. Examples of electrodes include anodes and cathodes, which may be used in a photosensitive optoelectronic device.

As used herein, the term “transparent” may refer to a material that permits at least 50% of the incident electromagnetic radiation in relevant wavelengths to be transmitted through it. In a photosensitive optoelectronic device, it may be desirable to allow the maximum amount of ambient electromagnetic radiation from the device exterior to be admitted to the photoconductive active interior region. That is, the electromagnetic radiation must reach a photoconductive layer(s), where it can be converted to electricity by photoconductive absorption. This often dictates that at least one of the electrical contacts or electrodes should be minimally absorbing and minimally reflecting of the incident electromagnetic radiation. In some cases, such a contact should be transparent or at least semi-transparent. In one embodiment, the transparent material may form at least part of an electrical contact or electrode.

As used herein, the term “semi-transparent” may refer to a material that permits some, but less than 50% transmission of ambient electromagnetic radiation in relevant wavelengths. Where a transparent or semi-transparent electrode is used, the opposing electrode may be a reflective material so that light which has passed through the cell without being absorbed is reflected back through the cell.

As used and depicted herein, a “layer” refers to a member or component of a device, for example an optoelectronic device, being principally defined by a thickness, for example in relation to other neighboring layers, and extending outward in length and width. It should be understood that the term “layer” is not necessarily limited to single layers or sheets of materials. In addition, it should be understood that the surfaces of certain layers, including the interface(s) of such layers with other material(s) or layers(s), may be imperfect, wherein said surfaces represent an interpenetrating, entangled or convoluted network with other material(s) or layer(s). Similarly, it should also be understood that a layer may be discontinuous, such that the continuity of said layer along the length and width may be disturbed or otherwise interrupted by other layer(s) or material(s).

As used herein, a “photoactive region” refers to a region of a device that absorbs electromagnetic radiation to generate excitons. Similarly, a layer is “photoactive” if it absorbs electromagnetic radiation to generate excitons. The excitons may dissociate into an electron and a hole in order to generate an electrical current.

As used herein, the terms “donor” and “acceptor” refer to the relative positions of the highest occupied molecular orbital (“HOMO”) and lowest unoccupied molecular orbital (“LUMO”) energy levels of two contacting but different organic materials. If the LUMO energy level of one material in contact with another is lower, then that material is an acceptor. Otherwise it is a donor. It is energetically favorable, in the absence of an external bias, for electrons at a donor-acceptor junction to move into the acceptor material, and for holes to move into the donor material.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

As used herein, the term “band gap” (E_{g}) of a polymer may refer to the energy difference between the HOMO and the LUMO. The band gap is typically reported in electron volts (eV). The band gap may be measured from the UV-vis spectroscopy or cyclic voltammetry. A “low band gap” polymer may refer to a polymer with a band gap below 2 eV, e.g., the polymer absorbs light with wavelengths longer than 620 nm.

As used herein, the term “excitation binding energy” (E_{B}) may refer to the following formula: E_{B}=(M⁺+M⁻)−(M*+M), where M⁺ and M⁻ are the total energy of a positively and negatively charged molecule, respectively; M* and M are the molecular energy at the first singlet state (S₁) and ground state, respectively. Excitation binding energy of acceptor or donor molecules affects the energy offset needed for efficient exciton dissociation. In certain examples, the escape yield of a hole increases as the HOMO offset increases. A decrease of exciton binding energy EB for the acceptor molecule leads to an increase of hole escape yield for the same HOMO offset between donor and acceptor molecules.

As used herein, “power conversion efficiency” (PCE) (η_{ρ}) may be expressed as:

$${η}_{ρ} = \frac{{V}_{OC} * F F * {J}_{SC}}{{P}_{O}}$$

wherein V_{OC} is the open circuit voltage, FF is the fill factor, J_{SC} is the short circuit current, and P_{O} is the input optical power.

As used herein, “spin coating” may refer to the process of solution depositing a layer or film of one material (i.e., the coating material) on a surface of an adjacent substrate or layer of material. The spin coating process may include applying a small amount of the coating material on the center of the substrate, which is either spinning at low speed or not spinning at all. The substrate is then rotated at high speed in order to spread the coating material by centrifugal force. Rotation is continued while the fluid spins off the edges of the substrate, until the desired thickness of the film is achieved. The applied solvent is usually volatile, and simultaneously evaporates. Therefore, the higher the angular speed of spinning, the thinner the film. The thickness of the film also depends on the viscosity and concentration of the solution and the solvent.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

The terms “halo,” “halogen,” and “halide” are used interchangeably and refer to fluorine, chlorine, bromine, and iodine.

The term “pseudohalogen” refers to polyatomic analogues of halogens, whose chemistry, resembling that of the true halogens, allows them to substitute for halogens in several classes of chemical compounds. Exemplary pseudohalogens include, but are not limited to, nitrile, cyaphide, isocyanide, cyanate, isocyanate, fulminate, thiocyanate, isothiocyanate, selenocyanate, tellurocyanate, azide, tetracarbonylcobaltate, trinitromethanide, and tricyanomethanide groups.

The term “acyl” refers to a substituted carbonyl radical (C(O)—Rₛ).

The term “ester” refers to a substituted oxycarbonyl (—O—C(O)—Rₛ or —C(O)—O—Rₛ) radical.

The term “ether” refers to an —ORₛ radical.

The terms “sulfanyl” or “thio-ether” are used interchangeably and refer to a —SRₛ radical.

The term “sulfinyl” refers to a —S(O)—Rₛ radical.

The term “sulfonyl” refers to a —SO₂—Rₛ radical.

The term “phosphino” refers to a —P(Rₛ)₃ radical, wherein each Rₛ can be same or different.

The term “silyl” refers to a —Si(Rₛ)₃ radical, wherein each Rₛ can be same or different.

The term “boryl” refers to a —B(Rₛ)₂ radical or its Lewis adduct —B(Rs)3 radical, wherein Rs can be same or different.

In each of the above, Rₛ can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof Preferred Rₛ is selected from the group consisting of alkyl, cycloalkyl, aryl, heteroaryl, and combination thereof.

The term “alkyl” refers to and includes both straight and branched chain alkyl radicals. Preferred alkyl groups are those containing from one to fifteen carbon atoms and includes methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl,and the like. Additionally, the alkyl group is optionally substituted.

The term “cycloalkyl” refers to and includes monocyclic, polycyclic, and spiro alkyl radicals. Preferred cycloalkyl groups are those containing 3 to 12 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group is optionally substituted.

The terms “heteroalkyl” or “heterocycloalkyl” refer to an alkyl or a cycloalkyl radical, respectively, having at least one carbon atom replaced by a heteroatom. Optionally the at least one heteroatom is selected from O, S, N, P, B, Si and Se, preferably, O, S or N. Additionally, the heteroalkyl or heterocycloalkyl group is optionally substituted.

The term “alkenyl” refers to and includes both straight and branched chain alkene radicals. Alkenyl groups are essentially alkyl groups that include at least one carbon-carbon double bond in the alkyl chain. Cycloalkenyl groups are essentially cycloalkyl groups that include at least one carbon-carbon double bond in the cycloalkyl ring. The term “heteroalkenyl” as used herein refers to an alkenyl radical having at least one carbon atom replaced by a heteroatom. Optionally the at least one heteroatom is selected from O, S, N, P, B, Si, and Se, preferably, O, S, or N. Preferred alkenyl, cycloalkenyl, or heteroalkenyl groups are those containing two to fifteen carbon atoms. Additionally, the alkenyl, cycloalkenyl, or heteroalkenyl group is optionally substituted.

The term “alkynyl” refers to and includes both straight and branched chain alkyne radicals. Preferred alkynyl groups are those containing two to fifteen carbon atoms. Additionally, the alkynyl group is optionally substituted.

The terms “aralkyl” or “arylalkyl” are used interchangeably and refer to an alkyl group that is substituted with an aryl group. Additionally, the aralkyl group is optionally substituted.

The term “heterocyclic group” refers to and includes aromatic and non-aromatic cyclic radicals containing at least one heteroatom. Optionally the at least one heteroatom is selected from O, S, N, P, B, Si, and Se, preferably, O, S, or N. Hetero-aromatic cyclic radicals may be used interchangeably with heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group may be optionally substituted.

The term “aryl” refers to and includes both single-ring aromatic hydrocarbyl groups and polycyclic aromatic ring systems. The polycyclic rings may have two or more rings in which two carbons are common to two adjoining rings (the rings are “fused”) wherein at least one of the rings is an aromatic hydrocarbyl group, e.g., the other rings can be cycloalkyls, cycloalkenyls, aryl, heterocycles, and/or heteroaryls. Preferred aryl groups are those containing six to thirty carbon atoms, preferably six to twenty carbon atoms, more preferably six to twelve carbon atoms. Especially preferred is an aryl group having six carbons, ten carbons or twelve carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, fluorene, and naphthalene. Additionally, the aryl group is optionally substituted.

The term “heteroaryl” refers to and includes both single-ring aromatic groups and polycyclic aromatic ring systems that include at least one heteroatom. The heteroatoms include, but are not limited to O, S, N, P, B, Si, and Se. In many instances, O, S, or N are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group is optionally substituted.

Of the aryl and heteroaryl groups listed above, the groups of triphenylene, naphthalene, anthracene, dibenzothiophene, dibenzofuran, dibenzoselenophene, carbazole, indolocarbazole, imidazole, pyridine, pyrazine, pyrimidine, triazine, and benzimidazole, and the respective aza-analogs of each thereof are of particular interest.

The terms alkyl, cycloalkyl, heteroalkyl, heterocycloalkyl, alkenyl, cycloalkenyl, heteroalkenyl, alkynyl, aralkyl, heterocyclic group, aryl, and heteroaryl, as used herein, are independently unsubstituted, or independently substituted, with one or more general substituents.

In many instances, the general substituents are selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acid, ether, ester, nitrile, isonitrile, sulfanyl, sulfanyl, sulfonyl, phosphino, and combinations thereof.

In some instances, the preferred general substituents are selected from the group consisting of deuterium, fluorine, alkyl, cycloalkyl, heteroalkyl, alkoxy, aryloxy, amino, silyl, alkenyl, cycloalkenyl, heteroalkenyl, aryl, heteroaryl, nitrile, isonitrile, sulfanyl, and combinations thereof.

In some instances, the preferred general substituents are selected from the group consisting of deuterium, fluorine, alkyl, cycloalkyl, alkoxy, aryloxy, amino, silyl, aryl, heteroaryl, sulfanyl, and combinations thereof.

In yet other instances, the more preferred general substituents are selected from the group consisting of deuterium, fluorine, alkyl, cycloalkyl, aryl, heteroaryl, and combinations thereof.

The terms “substituted” and “substitution” refer to a substituent other than H that is bonded to the relevant position, e.g., a carbon or nitrogen. For example, when R¹ represents mono-substitution, then one R¹ must be other than H (i.e., a substitution). Similarly, when R¹ represents di-substitution, then two of R¹ must be other than H. Similarly, when R¹ represents no substitution, R¹, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The “aza” designation in the fragments described herein, i.e. aza-dibenzofuran, aza-dibenzothiophene, etc. means that one or more of the C—H groups in the respective aromatic ring can be replaced by a nitrogen atom, for example, and without any limitation, azatriphenylene encompasses both dibenzo[f,h]quinoxaline and dibenzo[f,h]quinoline. One of ordinary skill in the art can readily envision other nitrogen analogs of the aza-derivatives described above, and all such analogs are intended to be encompassed by the terms as set forth herein.

As used herein, “deuterium” refers to an isotope of hydrogen. Deuterated compounds can be readily prepared using methods known in the art. For example, U.S. Pat. No. 8,557,400, Patent Pub. No. WO 2006/095951, and U.S. Pat. Application Pub. No. US 2011/0037057, which are hereby incorporated by reference in their entireties, describe the making of deuterium-substituted organometallic complexes. Further reference is made to Ming Yan, et al., Tetrahedron 2015, 71, 1425-30 and Atzrodt et al., Angew. Chem. Int. Ed. (Reviews) 2007, 46, 7744-65, which are incorporated by reference in their entireties, describe the deuteration of the methylene hydrogens in benzyl amines and efficient pathways to replace aromatic ring hydrogens with deuterium, respectively.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In some instance, a pair of adjacent substituents can be optionally joined or fused into a ring. The preferred ring is a five, six, or seven-membered carbocyclic or heterocyclic ring, includes both instances where the portion of the ring formed by the pair of substituents is saturated and where the portion of the ring formed by the pair of substituents is unsaturated. As used herein, “adjacent” means that the two substituents involved can be on the same ring next to each other, or on two neighboring rings having the two closest available substitutable positions, such as 2,2′ positions in a biphenyl, or 1,8 position in a naphthalene, as long as they can form a stable fused ring system.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD), such as described in U.S. Pat. No. 6,337,102 to Forrest et al., which is incorporated by reference in its entirety, and deposition by organic vapor jet printing (OVJP), such as described in U.S. Pat. No. 7,431,968, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and organic vapor jet printing (OVJP). Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons is a preferred range. Materials with asymmetric structures may have better solution processability than those having symmetric structures, because asymmetric materials may have a lower tendency to recrystallize. Dendrimer substituents may be used to enhance the ability of small molecules to undergo solution processing.

Devices fabricated in accordance with embodiments of the present invention may further optionally comprise a barrier layer. One purpose of the barrier layer is to protect the electrodes and organic layers from damaging exposure to harmful species in the environment including moisture, vapor and/or gases, etc. The barrier layer may be deposited over, under or next to a substrate, an electrode, or over any other parts of a device including an edge. The barrier layer may comprise a single layer, or multiple layers. The barrier layer may be formed by various known chemical vapor deposition techniques and may include compositions having a single phase as well as compositions having multiple phases. Any suitable material or combination of materials may be used for the barrier layer. The barrier layer may incorporate an inorganic or an organic compound or both. The preferred barrier layer comprises a mixture of a polymeric material and a non-polymeric material as described in U.S. Pat. No. 7,968,146, PCT Pat. Application Nos. PCT/US2007/023098 and PCT/US2009/042829, which are herein incorporated by reference in their entireties. To be considered a “mixture”, the aforesaid polymeric and non-polymeric materials comprising the barrier layer should be deposited under the same reaction conditions and/or at the same time. The weight ratio of polymeric to non-polymeric material may be in the range of 95:5 to 5:95. The polymeric material and the non-polymeric material may be created from the same precursor material. In one example, the mixture of a polymeric material and a non-polymeric material consists essentially of polymeric silicon and inorganic silicon.

Devices fabricated in accordance with embodiments of the invention can be incorporated into a wide variety of electronic component modules (or units) that can be incorporated into a variety of electronic products or intermediate components.

The materials and structures described herein may have applications in devices other than organic solar cells. For example, other optoelectronic devices such as organic electroluminescent devices (OLEDs) and organic photodetectors may employ the materials and structures. More generally, organic devices, such as organic transistors, may employ the materials and structures.

According to one aspect, the present disclosure relates to a compound of Formula I:

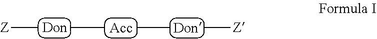

wherein Don and Don′ are each independently represented by Formula A or Formula B:

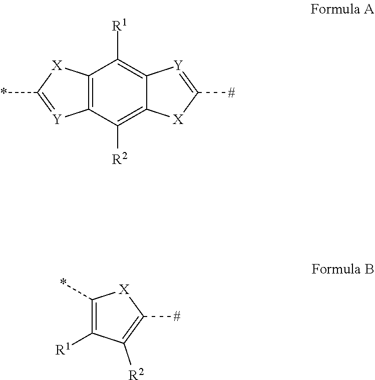

wherein, in Formula A and Formula B:
* represents the bond to Acc;
# represents the bond to Z or Z′;
each X is independently selected from the group consisting of O, S, Se, Te, GeRR′, CRR′, SiRR′, and NR.
each Y is independently selected from the group consisting of N and CR″.
R¹, R², R, R′, and R″ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and any two adjacent substituents optionally join to form a ring;
Acc is a divalent electron accepting group;
Z and Z′ are each together donating groups D and D′ respectively or accepting groups A and A′ respectively;
wherein each of Acc, Z, and Z′ may be further substituted with one or more substituents selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein any two adjacent substituents optionally join to form a ring.

In one embodiment, Acc is an electron accepting group selected from the group consisting of SO₂, CF₂, imine, ketone, polycyclic aromatic, polycyclic heteroaromatic, alkyl-borate, aryl-borate, alkoxy-borate, and combinations thereof;

In one embodiment, D and D′ are independently selected from the group consisting of alkoxy, aryloxy, heteroaryloxy, alkenyloxy, alkyloxy, allyloxy, amino, dialkylamino, diarylamino, thioalkyl, thioaryl, thioheteroaryl, and combinations thereof; and

In one embodiment, A and A′ are independently selected from the group consisting of halogen, NO₂, CN, SO₂R, CF₃, imine, ketone, aldehyde, polycyclic aromatic, polycyclic heteroaromatic, alkyl-borate, aryl-borate, alkoxy-borate, and combinations thereof; and

In one embodiment, each of Acc, Z, and Z′ may be further substituted with one or more substituents selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; wherein any two adjacent substituents optionally join to form a ring.

In one embodiment, the compound is represented by Formula II:

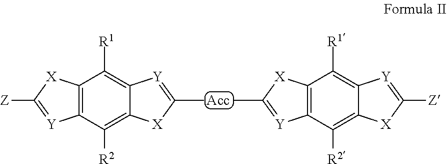

wherein, in Formula II,
Acc is a divalent electron accepting group selected from the group consisting of polycyclic aromatic, polycyclic heteroaromatic, aryl-borate, alkoxy-borate, and combinations thereof;
each X is independently selected from the group consisting of O, S, Se, Te, GeRR′, CRR′, SiRR′, and NR;
each Y is independently selected from the group consisting of N and CR″.
Z and Z′ are each together donating groups D and D′ respectively or accepting groups A and A′ respectively;
D and D′ are independently selected from the group consisting of alkoxy, aryloxy, heteroaryloxy, alkenyloxy, alkyloxy, allyloxy, amino, dialkylamino, diarylamino, thioalkyl, thioaryl, thioheteroaryl, and combinations thereof;
A and A′ are independently selected from the group consisting of halogen, NO₂, CN, SO₂R, CF₃, imine, ketone, aldehyde, polycyclic aromatic, polycyclic heteroaromatic, alkyl-borate, aryl-borate, alkoxy-borate, and combinations thereof;
R¹, R^{1′}, R², R^{2′}, R, R′, and R″ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
any two adjacent substituents optionally join to form a ring.

In one embodiment, at least one of R¹ and R² is represented by one of the following structures:

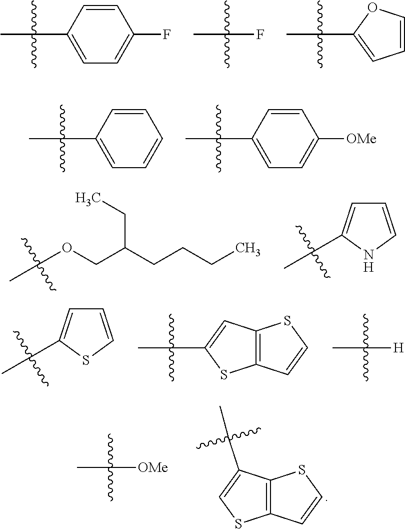

In one embodiment, Acc is represented by one of the following structures:

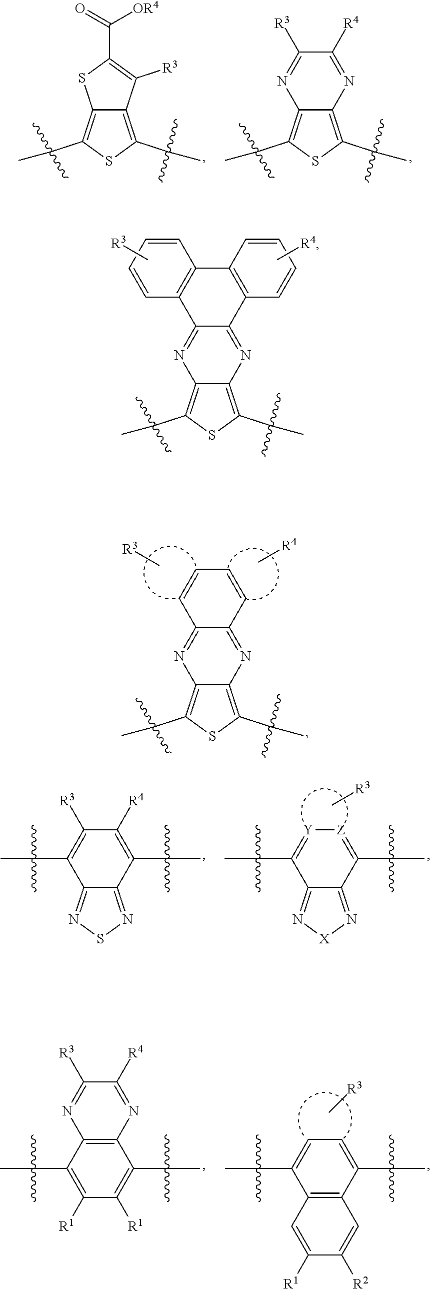

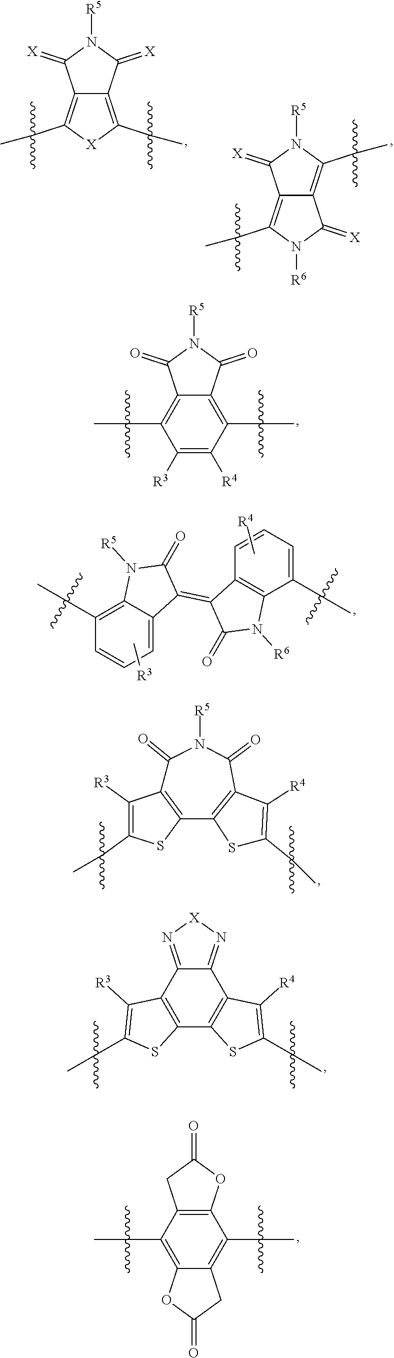

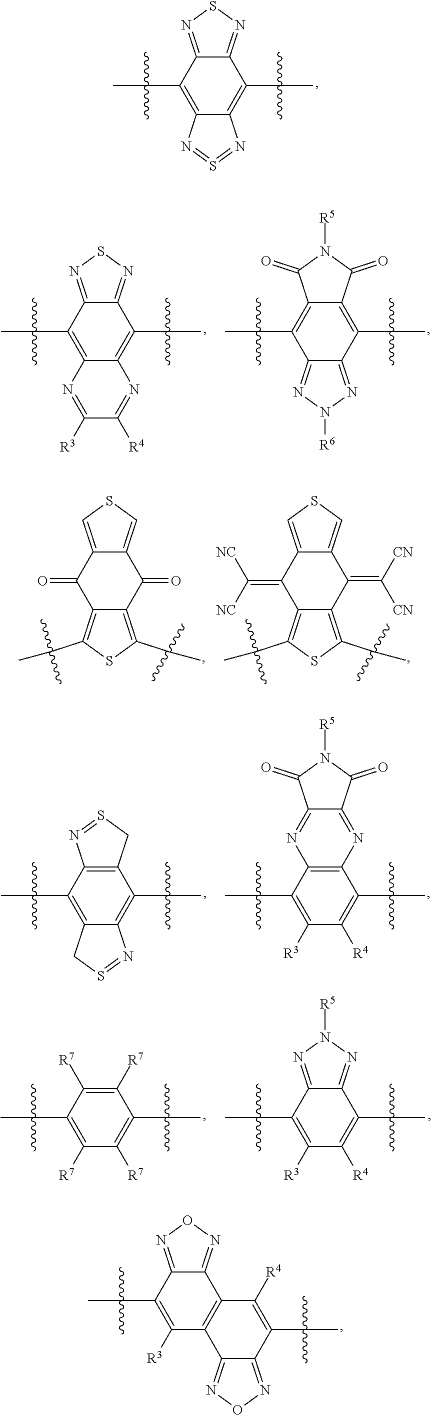

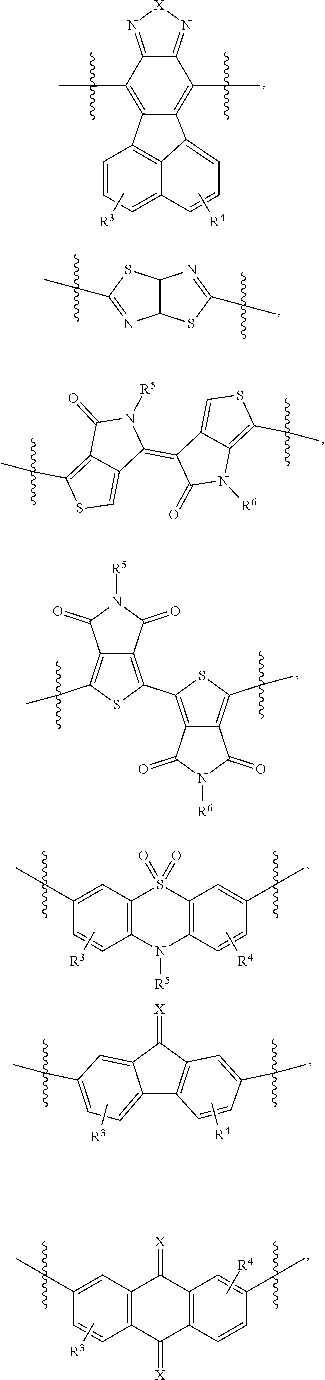

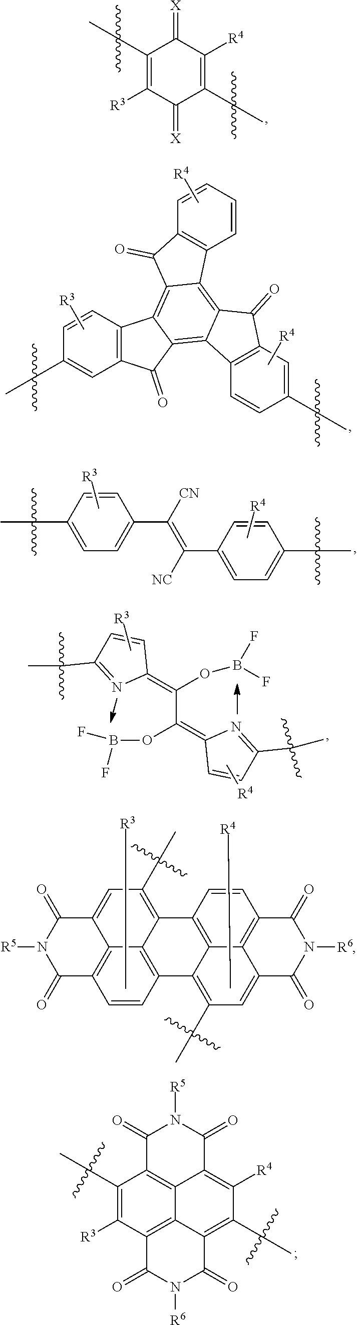

wherein wavy lines indicate bonds to Don and Don′;
wherein each X independently represents O, S, Se, NR⁵, or C(CN)₂;
wherein Y and Z independently represent CR⁴ and N;
wherein [symbol] represents optional aryl, heteroaryl, polyaromatic, or polyheteroaryl ring fusions;
wherein R³, R⁴, R⁵, and R⁶ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein each R⁷ is independently an electron-withdrawing group selected from the group consisting of halogen, haloalkyl, aryl, heteroaryl, nitrile, and combinations thereof; and
wherein any two adjacent substituents optionally join to form a ring.

In one embodiment, Z and Z′ are independently represented by one of the following structures:

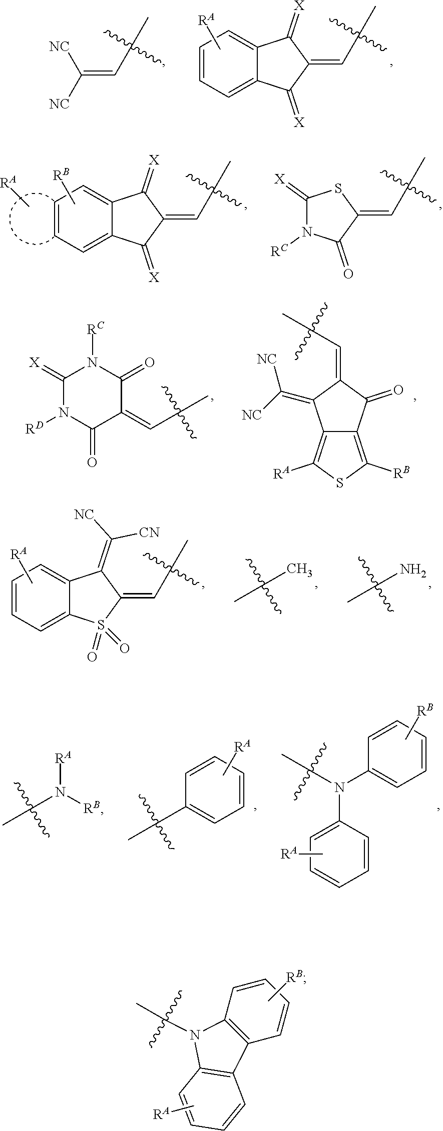

wherein each X independently represents O, S, Se, NR^{C}, or C(CN)₂;
R^{A}, R^{B}, R^{C}, and R^{D} independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein any two adjacent substituents optionally join to form a ring.

In one embodiment, Z and Z′ are each an acceptor group selected from the group consisting of:

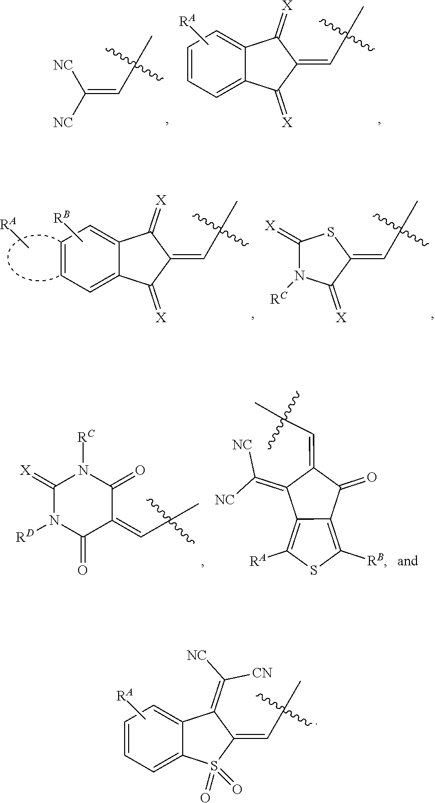

In one embodiment, Z and Z′ are each a donor group selected from the group consisting of:

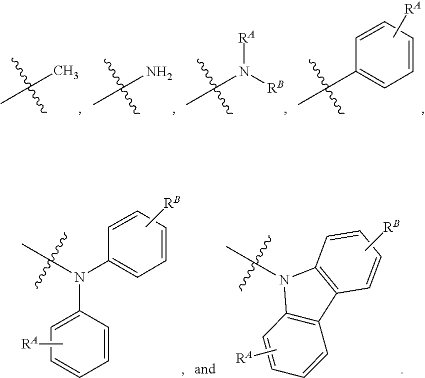

In one embodiment, Acc is selected from the group consisting of:

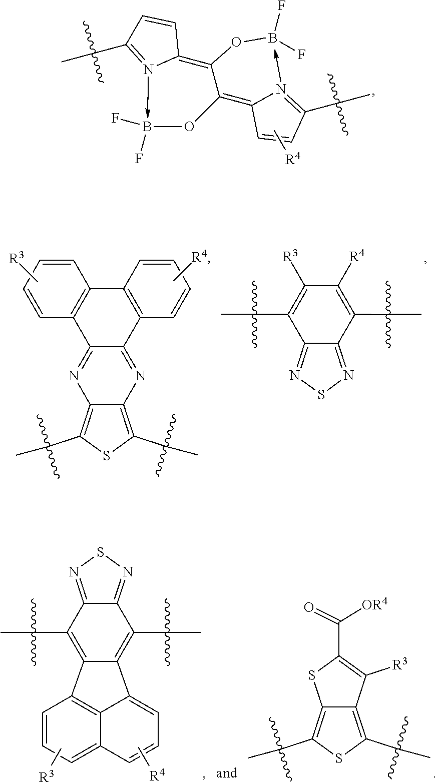

In one embodiment, the compound is represented by one of the following structures:

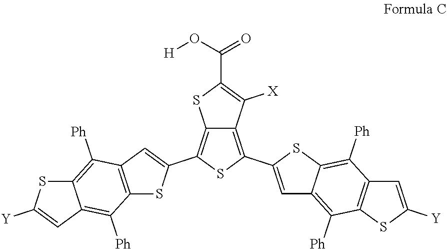

wherein, in Formula C:
X is F, CF₃, CN, SO₃H, or SO₂Me; and
Y is NH₂, NMe₂, NPh₂, N(4-OMePh)₂, N(3,4,5-(OMe)Ph)₂, or N(2,4,6-(OMe)Ph)₂;

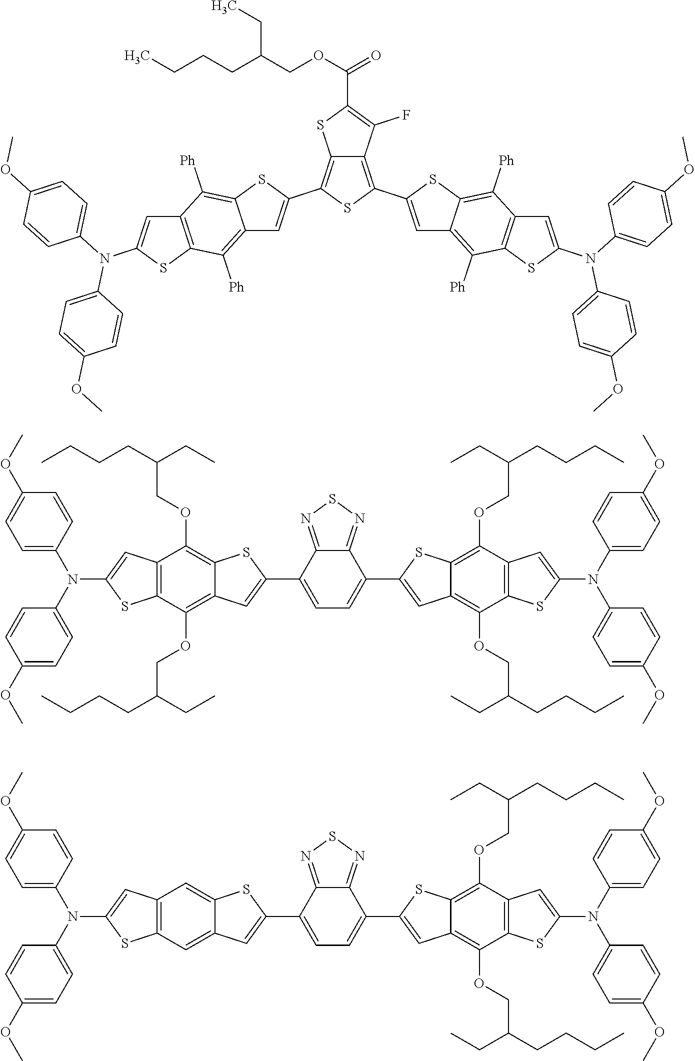

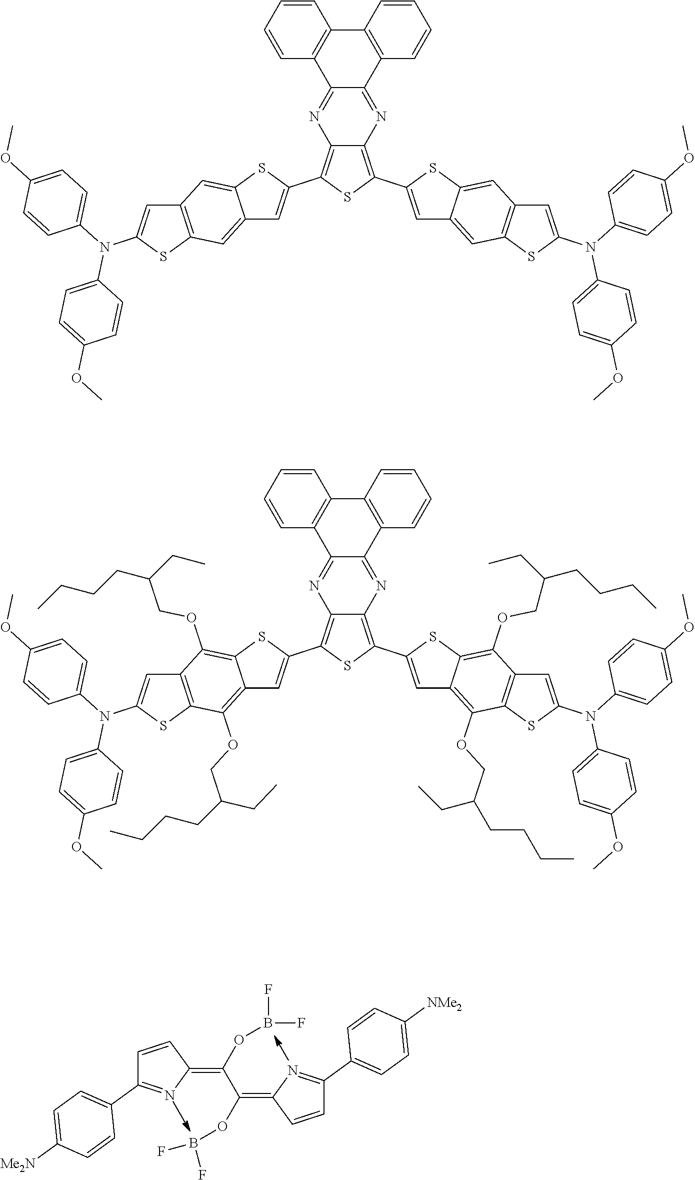 [SMILES: COc1ccc(N(c2ccc(OC)cc2)c2cc3cc4sc(-c5sc(-c6cc7cc8sc(N(c9ccc(OC)cc9)c9ccc(OC)cc9)cc8cc7s6)c6nc7c8ccccc8c8ccccc8c7nc56)cc4cc3s2)cc1 ; CCCCC(CC)COc1c2cc(N(c3ccc(OC)cc3)c3ccc(OC)cc3)sc2c(OCC(CC)CCCC)c2cc(-c3sc(-c4cc5c(OCC(CC)CCCC)c6sc(N(c7ccc(OC)cc7)c7ccc(OC)cc7)cc6c(OCC(CC)CCCC)c5s4)c4nc5c6ccccc6c6ccccc6c5nc34)sc12 ; CN(C)c1ccc(C2=N/C(=C(OB(F)F)\C(OB(F)F)=C3\C=CC(c4ccc(N(C)C)cc4)=N3)C=C2)cc1]

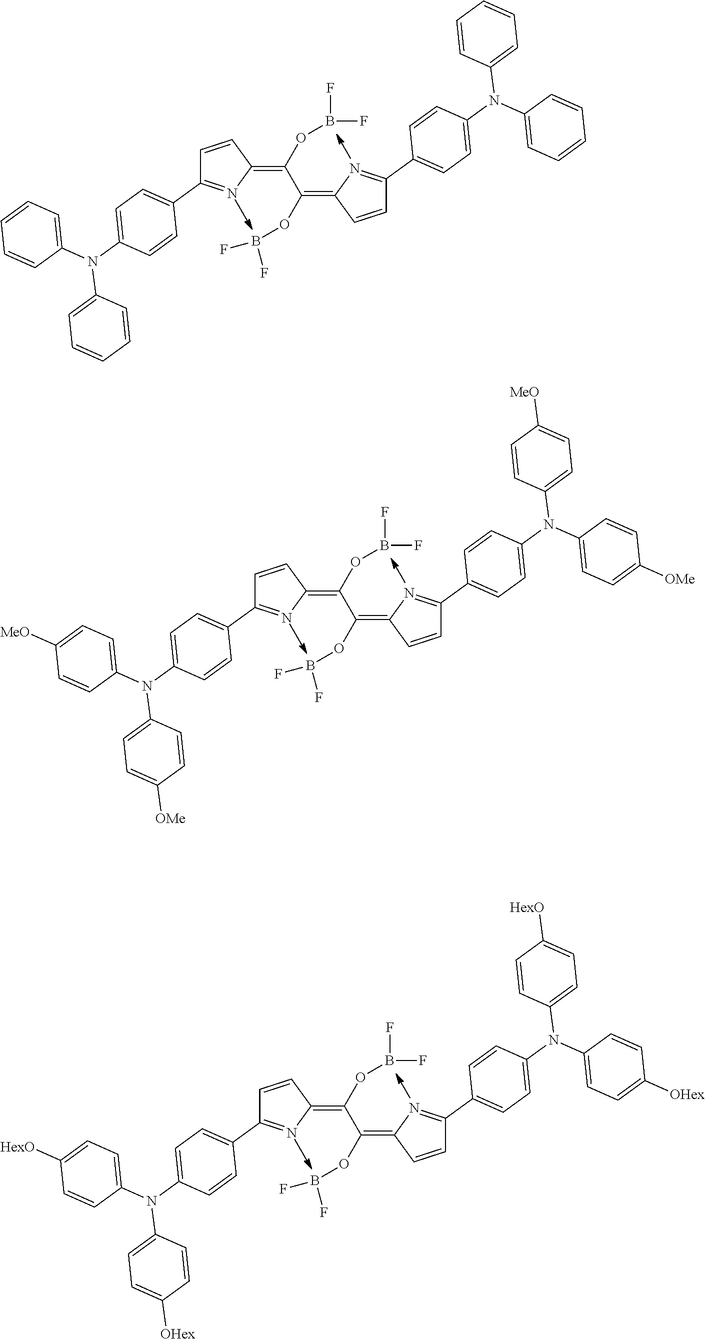

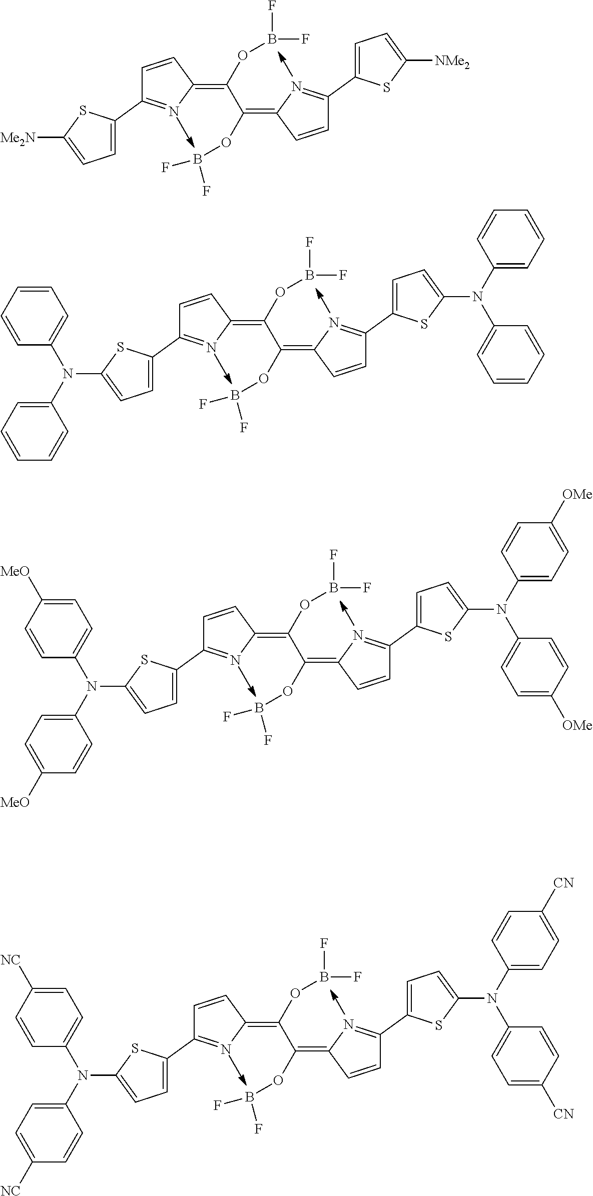 [SMILES: CN(C)c1ccc(C2=N/C(=C(OB(F)F)\C(OB(F)F)=C3\C=CC(c4ccc(N(C)C)s4)=N3)C=C2)s1 ; FB(F)OC(=C1\C=CC(c2ccc(N(c3ccccc3)c3ccccc3)s2)=N1)/C(OB(F)F)=C1/C=CC(c2ccc(N(c3ccccc3)c3ccccc3)s2)=N1 ; COc1ccc(N(c2ccc(OC)cc2)c2ccc(C3=N/C(=C(OB(F)F)\C(OB(F)F)=C4\C=CC(c5ccc(N(c6ccc(OC)cc6)c6ccc(OC)cc6)s5)=N4)C=C3)s2)cc1 ; N#Cc1ccc(N(c2ccc(C#N)cc2)c2ccc(C3=N/C(=C(OB(F)F)\C(OB(F)F)=C4\C=CC(c5ccc(N(c6ccc(C#N)cc6)c6ccc(C#N)cc6)s5)=N4)C=C3)s2)cc1]

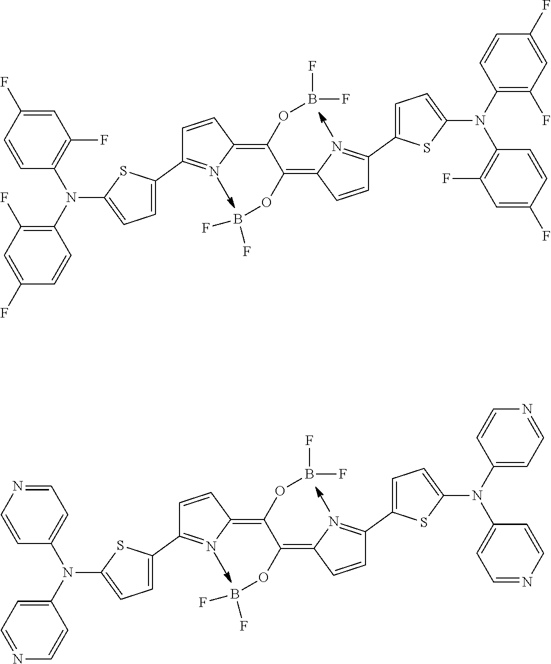 [SMILES: FB(F)OC(=C1\C=CC(c2ccc(N(c3ccc(F)cc3F)c3ccc(F)cc3F)s2)=N1)/C(OB(F)F)=C1/C=CC(c2ccc(N(c3ccc(F)cc3F)c3ccc(F)cc3F)s2)=N1 ; FB(F)OC(=C1\C=CC(c2ccc(N(c3ccncc3)c3ccncc3)s2)=N1)/C(OB(F)F)=C1/C=CC(c2ccc(N(c3ccncc3)c3ccncc3)s2)=N1]

According to another aspect, a formulation comprising a compound described herein is also disclosed.

Organic Photovoltaic Cells

In one aspect, the invention relates to an OPV device comprising a compound of the disclosure. In one embodiment, the OPV device includes an anode; a cathode; and an active material positioned between the anode and cathode, wherein the active material comprises an acceptor and a donor.

In one embodiment, the OPV device comprises a single junction organic photovoltaic device. In one embodiment, the OPV device comprises two electrodes having an anode and a cathode in superposed relation, at least one donor composition, and at least one acceptor composition, wherein the donor-acceptor material or active layer is positioned between the two electrodes. In one embodiment, one or more intermediate layers may be positioned between the anode and the active layer. Additionally, or alternatively, one or more intermediate layers may be positioned between the active layer and cathode.

In one embodiment, the anode comprises a conducting oxide, thin metal layer, or conducting polymer. In one embodiment, the anode comprises a conductive metal oxide. Exemplary conductive metal oxides include, but are not limited to, indium tin oxide (ITO), tin oxide (TO), gallium indium tin oxide (GITO), zinc oxide (ZO), and zinc indium tin oxide (ZITO). In one embodiment, the anode comprises a metal layer. Exemplary metals for the metal layer include, but are not limited to, Ag, Au, Pd, Pt, Ti, V, Zn, Sn, Al, Co, Ni, Cu, Cr, and combinations thereof. In one embodiment, the metal layer comprises a thin metal layer. In one embodiment, the anode 102 comprises a conductive polymer. Exemplary conductive polymers include, but are not limited to, polyanaline (PANI), or 3,4-polyethyl-enedioxythiophene:polystyrenesulfonate (PEDOT:PSS). In one embodiment, thickness of the anode is between about 0.1-100 nm. In one embodiment, thickness of the anode is between about 1-10 nm. In one embodiment, thickness of the anode is between about 0.1-10 nm. In one embodiment, thickness of the anode is between about 10-100 nm. In one embodiment, anode comprises a transparent or semi-transparent conductive material.

In one embodiment, the cathode comprises a conducting oxide, a metal layer, or conducting polymer. Exemplary conducting oxide, metal layers, and conducting polymers are described elsewhere herein. In one embodiment, the cathode comprises a thin metal layer. In one embodiment, the cathode comprises a metal or metal alloy. In one embodiment, the cathode may comprise Ca, Al, Mg, Ti, W, Ag, Au, or another appropriate metal, or an alloy thereof. In one embodiment, the thickness of the cathode is between about 0.1-100 nm. In one embodiment, the thickness of the cathode is between about 1-10 nm. In one embodiment, the thickness of the cathode is between about 0.1-10 nm. In one embodiment, the thickness of the cathode is between about 10-100 nm. In one embodiment, cathode comprises a transparent or semi-transparent conductive material.

In one embodiment, the OPV device may comprise one or more charge collecting/transporting intermediate layers positioned between an electrode and the active region or layer. In one embodiment, the OPV device comprises one or more intermediate layers. In one embodiment, the intermediate layer comprises a metal oxide. Exemplary metal oxides include, but are not limited to, MoO₃, MoOₓ, V₂O₅, ZnO, and TiO₂. In one embodiment, the first intermediate layer has the same composition as the second intermediate layer. In one embodiment, the first intermediate layer and the second intermediate layer have different compositions. In one embodiment, the thickness of the intermediate layers are each independently between about 0.1-100 nm. In one embodiment, the thickness of the intermediate layers are each independently between about 1-10 nm. In one embodiment, the thickness of the intermediate layers are each independently between about 0.1-10 nm. In one embodiment, the thickness of the intermediate layers are each independently between about 10-100 nm.

In one embodiment, the OPV device comprises various layers of a tandem or multi junction photovoltaic device. In one embodiment, the OPV device comprises two electrodes having an anode and a 204 in superposed relation, at least one donor composition, and at least one acceptor composition positioned within a plurality of active layers or regions between the two electrodes. Additional active layers or regions are also possible. In one embodiment, the anode and the cathode each independently comprise a conducting oxide, thin metal layer, or conducting polymer. Exemplary conducting oxides, metal layers, and conducting polymers are described elsewhere herein.

In one embodiment, the OPV device comprises one or more intermediate layers positioned between the anode and a first active layer. Additionally, or alternatively, at least one intermediate layer may be positioned between the second active layer and cathode. In one embodiment, the OPV device comprises one or more intermediate layers positioned between the first active layer and the second active layer. In one embodiment, the OPV device comprises a first intermediate layer. In one embodiment, the OPV device comprises a second intermediate layer. In one embodiment, the OPV device comprises a third intermediate layer. In one embodiment, the OPV device comprises both first and second intermediate layers. In one embodiment, the OPV device comprises both first and third intermediate layers. In one embodiment, the OPV device comprises both second and third intermediate layers. In one embodiment, the OPV device comprises first, second, and third intermediate layers. In one embodiment, the first, second, and/or third intermediate layer comprises a metal oxide. Exemplary metal oxides are described elsewhere herein.

### EXPERIMENTAL EXAMPLES

Small molecules are more reliable materials for organic photovoltaics (OPVs) compared to polymers, due to their reproducible synthesis and facile purification. Especially for device fabrication involving vapor deposition techniques, small molecules are obvious candidates compared to polymers.

Small molecules with acceptor-donor-acceptor (ADA) type triad, where two acceptor molecules are covalently linked to either end of donor molecules, are extensively used in high efficiency OPV devices (with PCE of ˜17%). IT-IC, IT-4F, m-ITIC molecules are some of those to name a few of these ADA type molecules in high efficiency devices. On the other hand, OPVs with donor-acceptor-donor (DAD) type of molecules are relatively less explored. The current efficiency of OPV devices with DAD type molecules are also relatively low compared to that with ADA type molecules. Here we are trying to develop new type of DAD type small molecule for semitransparent organic photovoltaics.

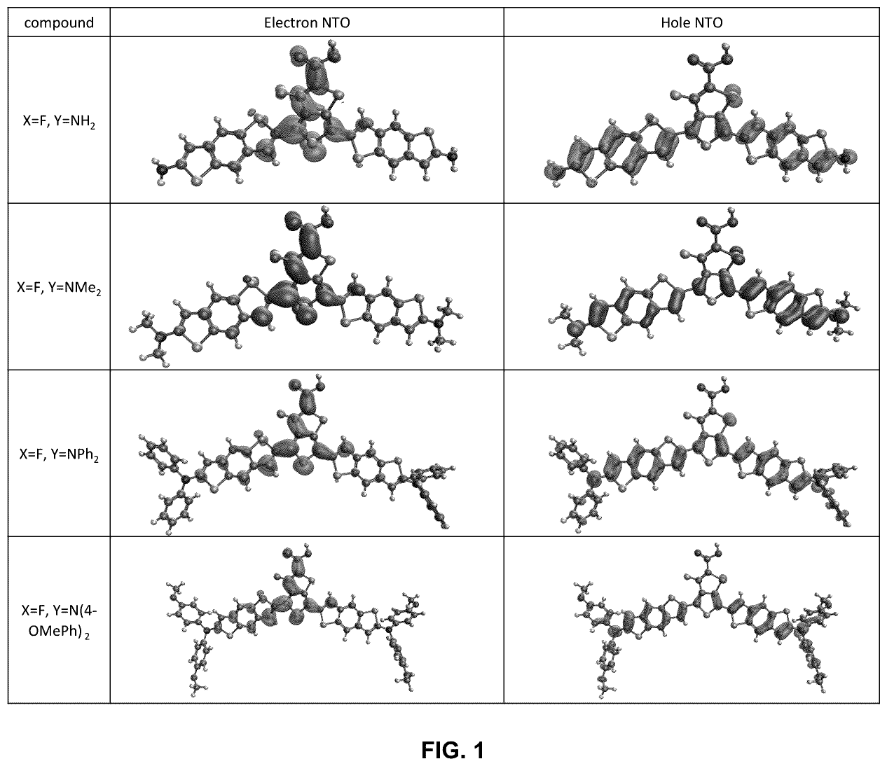

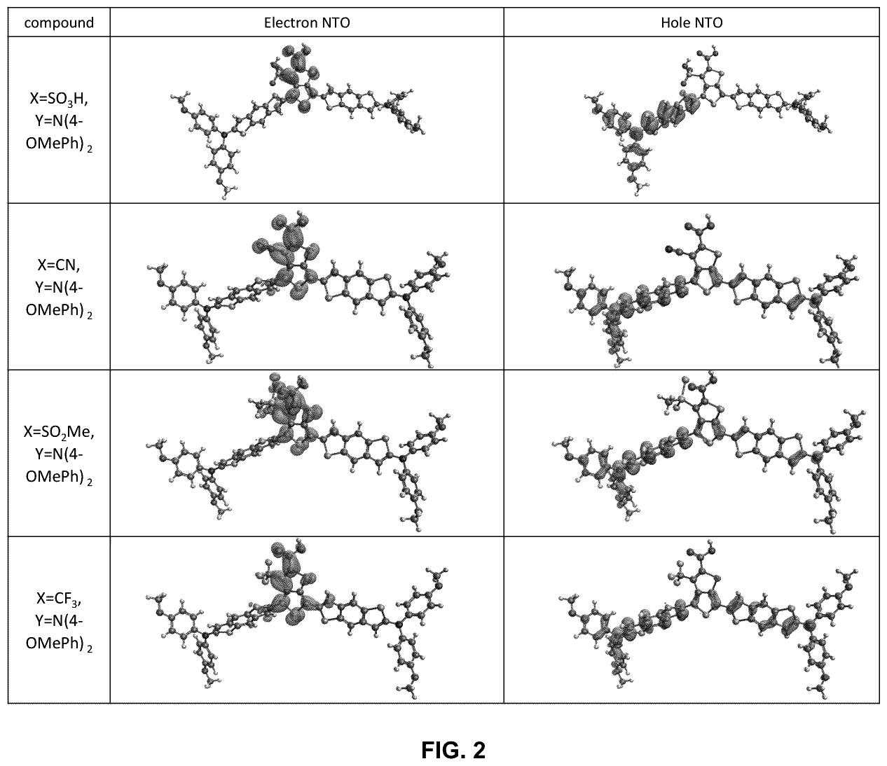

PCE-10 polymers such as 1 are used in some high efficiency semitransparent OPV devices. It consists of monomer unit with benzodithiophene (BDT) derivative linked to thienothiophene unit. The compounds described herein are related to monomer analogues of the PCE-10 polymer. The monomer analogues can be represented by the simplified structure 2, which consist of thienothiophene with BDT unit linked to the either side of thiophene at 2 and 5 positions and resembles donor-acceptor-donor type moiety. To determine the optimal structure with NIR absorption, DFT/TD-DFT calculation was performed on structure 2 with varying functional group X and Y (Table 1, FIG. 1, and FIG. 2). In structure 2, X and Y represent the group with electron donating and electron withdrawing nature, respectively, to maintain the donor-acceptor-donor (DAD) feature. DFT/TD-DFT calculations helped to optimize the synthetically feasible structure, identifying electron withdrawing unit X as fluorine and electron donating unit Y as bis(4-methoxy) diphenyl amine group as the most beneficial groups.

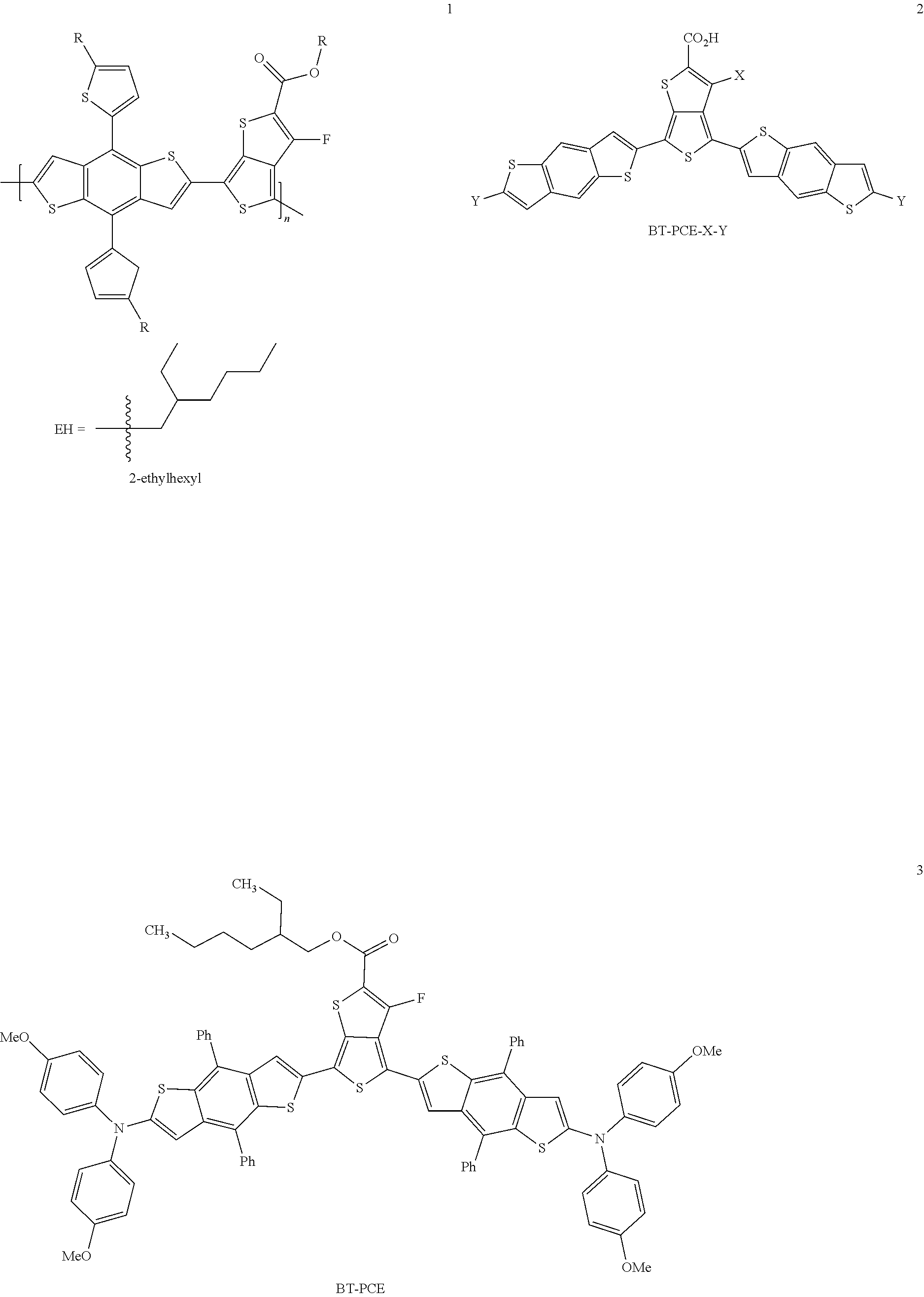

TABLE 1
DFT/ TD-DFT calculation for BT-PCE-X-Y -structures and CV data for BT-PCE

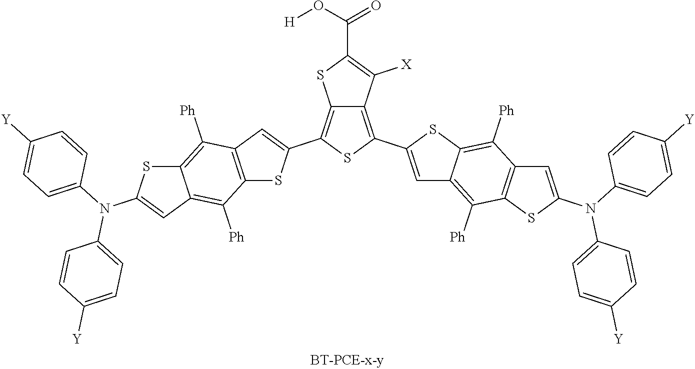

| | Theoretical data for S₁ state | | | | | From CV data | | |
|---|---|---|---|---|---|---|---|---|
| | | | | | HOMO/ | | | HOMO/ |
| | λ (eV, | | HOMO | LUMO | LUMO | HOMO | LUMO | LUMO |
| BT-PCE-X-Y | nm) | ƒ | (eV) | (eV) | gap | (eV) | (eV) | gap |
| BT-PCE | — | — | — | — | — | -4.93 | -2.65 | 2.28 |
| X = F, Y = NH₂ | 2.72, 455 | 0.74 | −4.78 | −2.39 | 2.39 | — | — | — |
| X = F, | 2.16, 574 | 1.12 | −4.62 | −2.23 | 2.39 | — | — | — |
| Y = NMe₂ | | | | | | | | |
| X = F, | 2.11, 587 | 1.46 | −4.79 | −2.39 | 2.4 | — | — | — |
| Y = NPh₂ | | | | | | | | |
| X = F, Y = N(4- | 1.97, 629 | 1.45 | −4.54 | −2.31 | 2.23 | — | — | — |
| OMePh)₂ | | | | | | | | |
| X = SO₃H, | 1.58, 785 | 0.25 | −4.62 | −2.74 | 1.88 | — | — | — |
| Y = N(4- | | | | | | | | |
| OMePh)₂ | | | | | | | | |
| X = CN, | 1.77, 700 | 0.47 | −4.65 | −2.56 | 2.09 | — | — | — |
| Y = N(4- | | | | | | | | |
| OMePh)₂ | | | | | | | | |
| X = SO₂Me, | 1.83, 673 | 0.35 | −4.68 | −2.53 | 2.15 | — | — | — |
| Y = N(4- | | | | | | | | |
| OMePh)₂ | | | | | | | | |
| X = CF₃, | 1.99, 623 | 0.69 | −4.62 | −2.34 | 2.28 | — | — | — |
| Y = N(4- | | | | | | | | |
| OMePh)₂ | | | | | | | | |
| X = F, | 2.02, 613 | 1.49 | −4.65 | −2.37 | 2.28 | — | — | — |
| Y = N[3,4,5(4- | | | | | | | | |
| OMe) Ph]₂ | | | | | | | | |
| X = F, | 2.01, 617 | 1.52 | −4.13 | −1.93 | 2.2 | — | — | — |
| Y = N[2,4,6(4- | | | | | | | | |
| OMe) Ph]₂ | | | | | | | | |

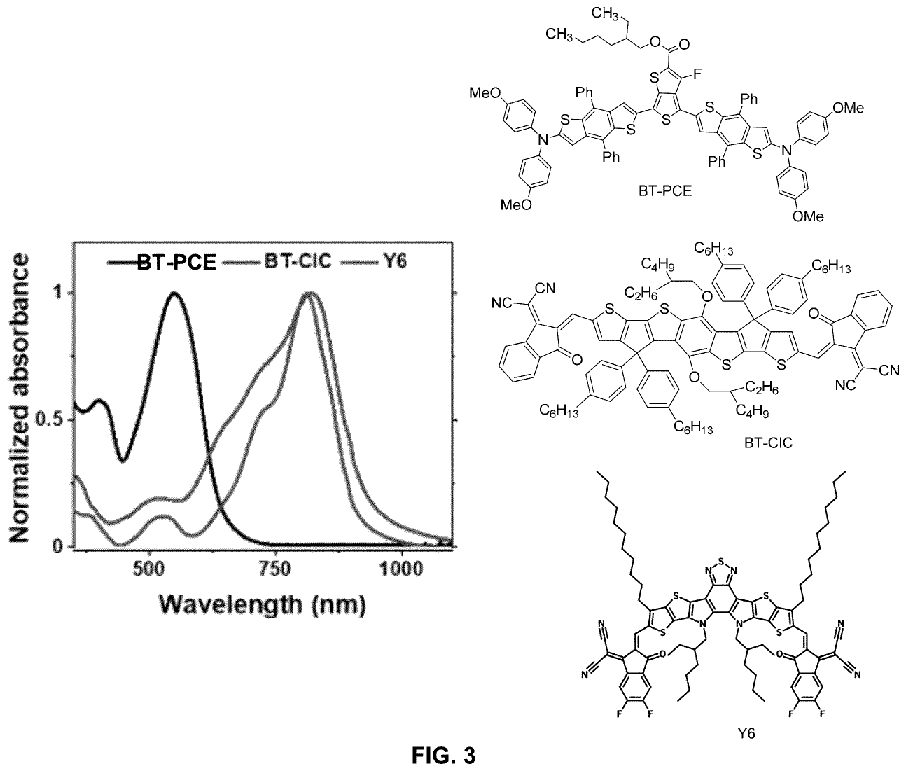

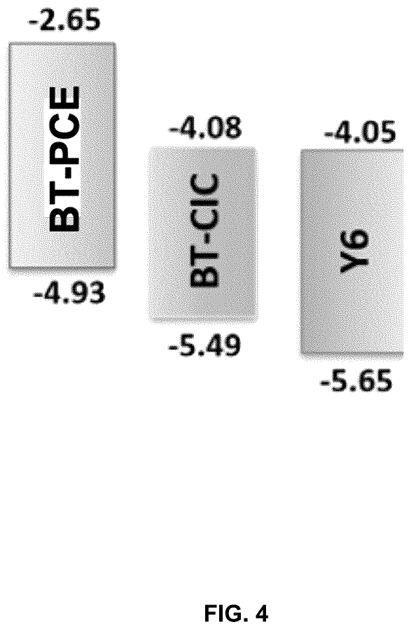

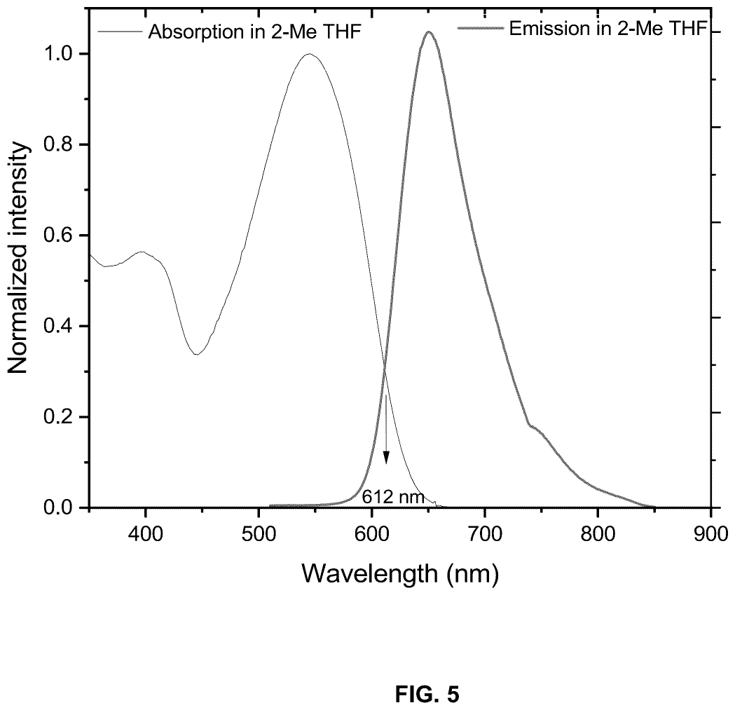

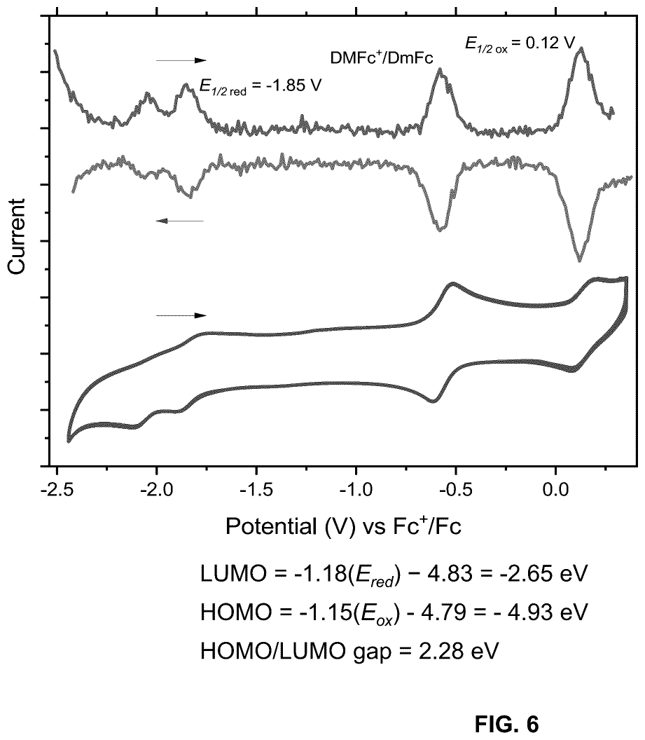

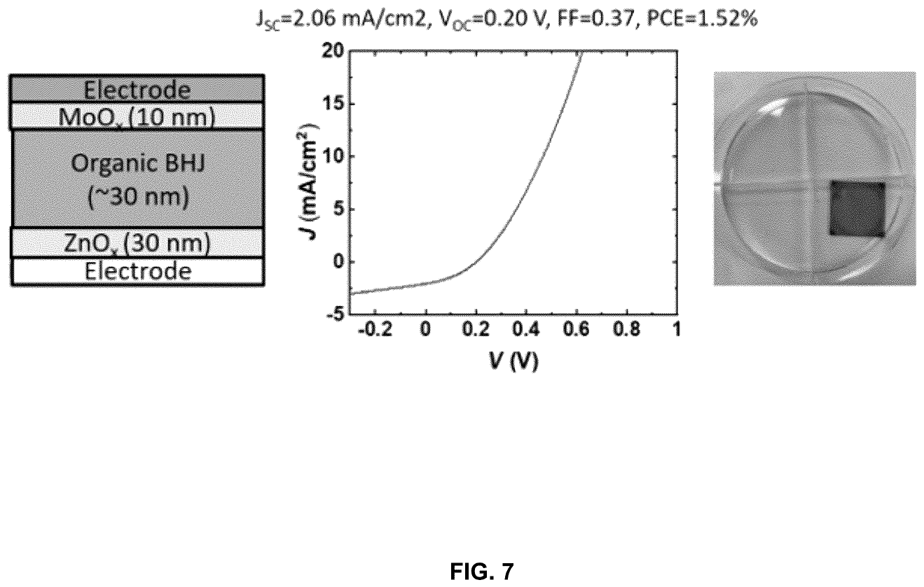

The computational and experimental data in Table 1, FIG. 1, and FIG. 2 support the following structures as donor materials for organic photovoltaics. BT-PCE (3) was synthesized and its photophysical data obtained. BT-PCE exhibited UV-Vis absorbance with absorbance maxima at 550 nm, tailing around 750 nm (FIG. 3) with optical band gap of 2.02 eV and electrochemical band gap of 2.28 eV (FIG. 4). The UV-Vis absorption and emission spectra of BT-PCT is given in FIG. 5. A cyclic voltammogram (CV) of BT-PCE is given in FIG. 6. On device fabrication with BT-PCE donor and Y6 acceptor, it showed a power conversion efficiency (PCE) of 1.52% (FIG. 7).

BT-PCE was synthesized using a commercially available thienothiophene acceptor unit, 2-ethyl hexyl 4,6-dibromo-3-fluorothieno[3,4-b]thiophene-2-carboxylate, which was then subjected to Stille coupling with benzodithiophene trimethyl stanyl derivative.

To further increase the diversity of DAD molecules for semitransparent OPV with better power conversion efficiency, the central acceptor unit of BT-PCE was modified. The following structural moieties 4a-4g were chosen for DFT calculation to screen structures for acceptor unit, with the goal of identifying the acceptor with the lowest LUMO energy level. The DFT calculations showed that benzothiadiazole 4e and 9,10-phenanthrenequinone thieno pyrazine 4f are better acceptors with lowest LUMO levels −2.34 eV and −2.50 eV respectively (Table 2).

TABLE 2
Chemical structure of acceptor molecules chosen for DFT calculations and the corresponding
HOMO LUMO values.

| | HOMO | | | HOMO | |
|---|---|---|---|---|---|
| Compound | (eV) | LUMO (eV) | Compound | (eV) | LUMO (eV) |
| 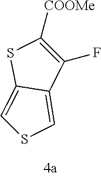 [SMILES: COC(=O)c1sc2cscc2c1F] | −5.98 | −1.96 | 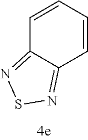 | −6.61 | −2.34 |
| 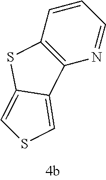 [SMILES: c1cnc2c(c1)sc1cscc12] | −5.71 | −1.36 | 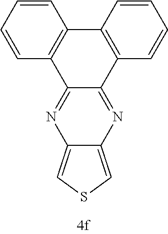 [SMILES: c1ccc2c(c1)c1ccccc1c1nc3cscc3nc21] | −5.77 | −2.50 |
| 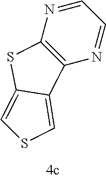 [SMILES: c1cnc2c(n1)sc1cscc12] | −5.98 | −1.85 | 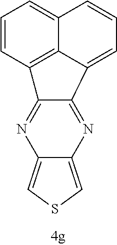 [SMILES: c1cc2c3c(cccc3c1)-c1nc3cscc3nc1-2] | −5.74 | −2.14 |
| 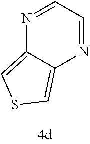 | −5.98 | −2.28 | | | |

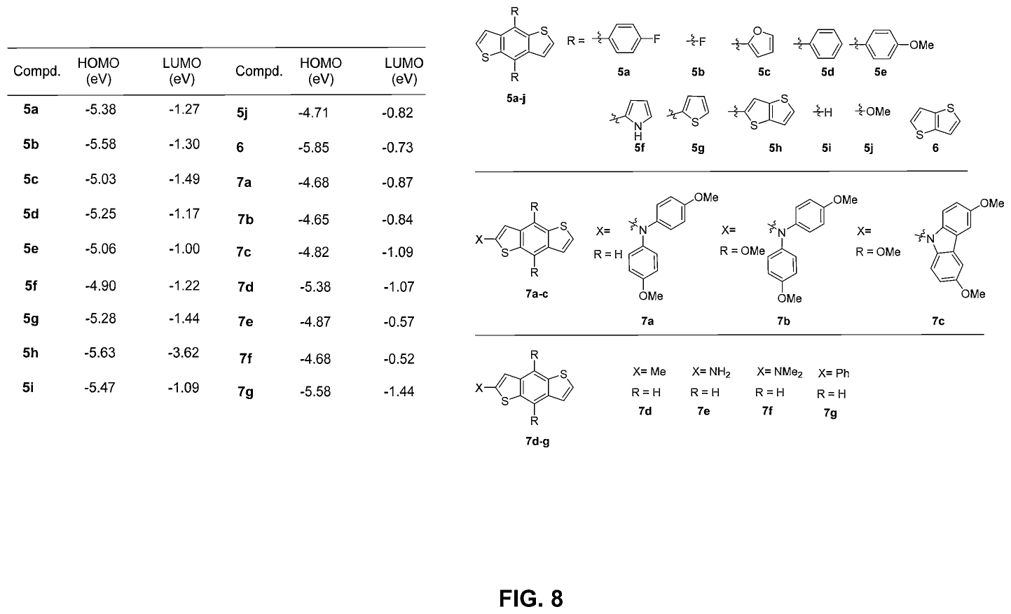

Similarly, DFT calculation of the donor component (FIG. 8) revealed BDT unit 7b as the donor molecule with the highest HOMO energy level (−4.65 eV).

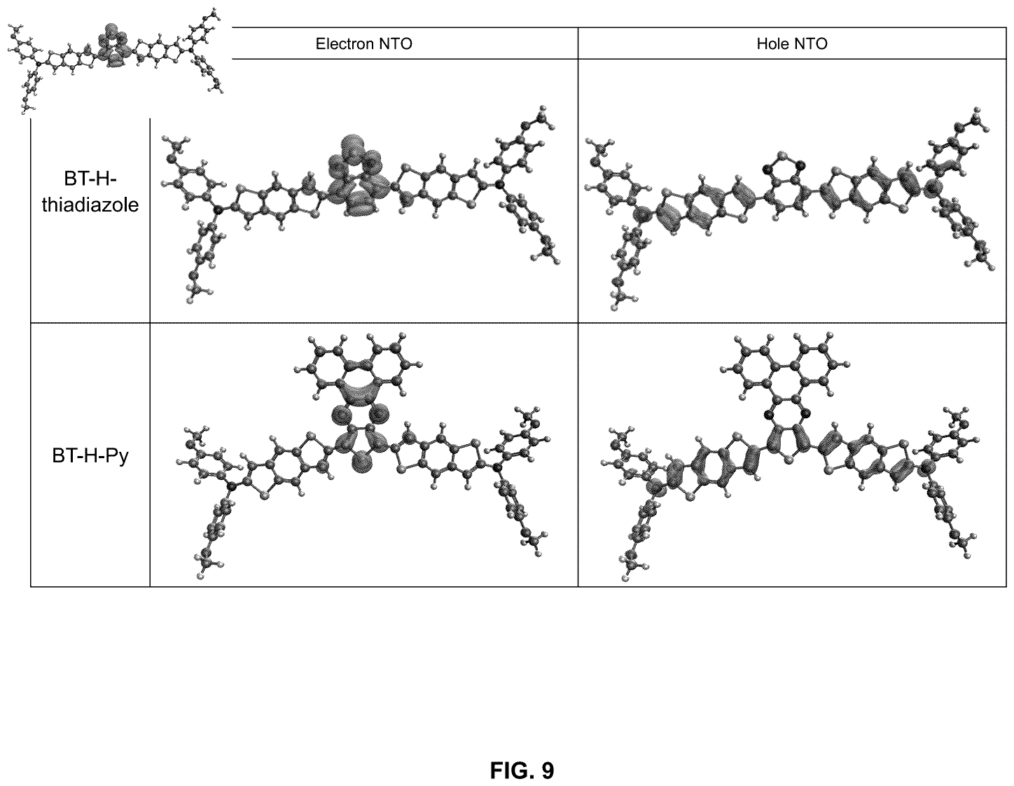

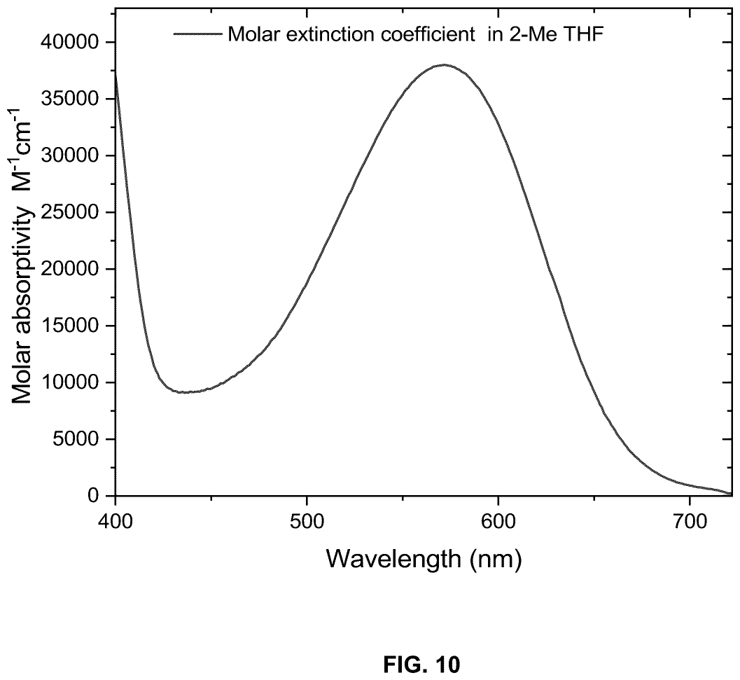

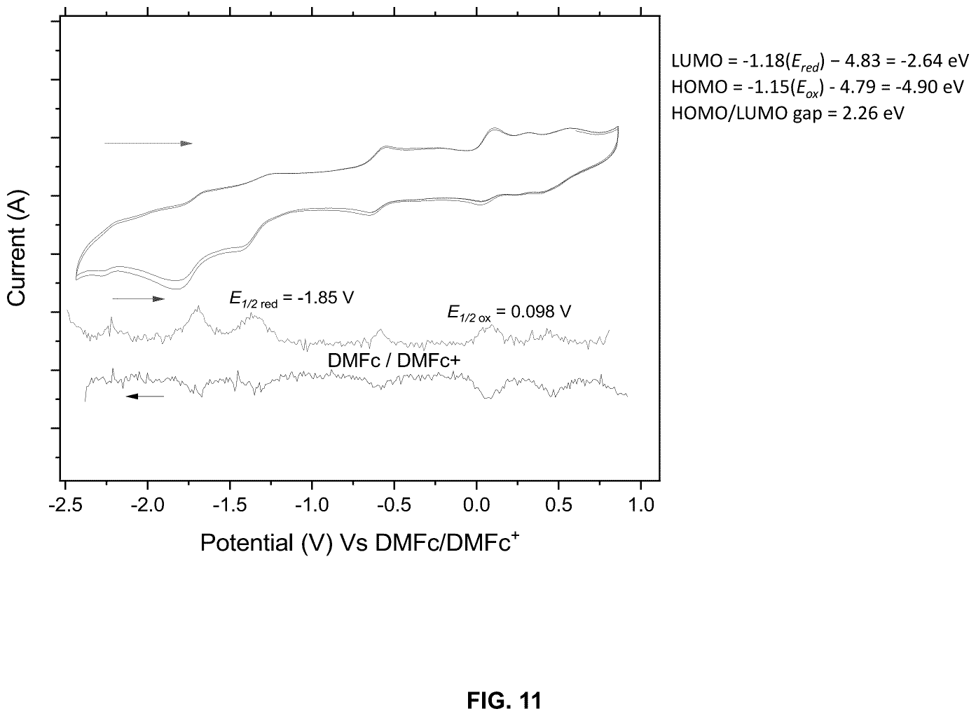

Based on these DFT calculations, DAD-type molecules were synthesized with donor unit 7b tethered to the acceptor cores 4e and 4f. DFT/TDDFT calculations were performed on these systems separately. BT-Thiadiazole 9 and BT-Py 11 were synthesized. HOMO/LUMO values were computed by DFT calculation and electrochemical methods (Table 3, Table 4, and FIG. 9) . The UV-Vis absorption spectrum of BT-thiadiazole is showin in FIG. 10. The CV of BT-thiadiazole is shown in FIG. 11.

TABLE 3
Chemical structure and DFT/ TD-DFT calculation for BT-thiadiazole, BT-H-thiadiazole and CV
data for BT-thiadiazole.

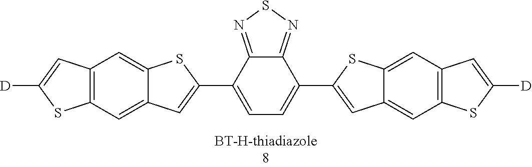 [SMILES: [2H]c1cc2cc3sc(-c4ccc(-c5cc6cc7sc([2H])cc7cc6s5)c5nsnc45)cc3cc2s1]

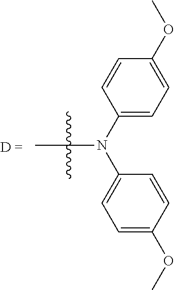 [SMILES: *N(c1ccc(OC)cc1)c1ccc(OC)cc1]

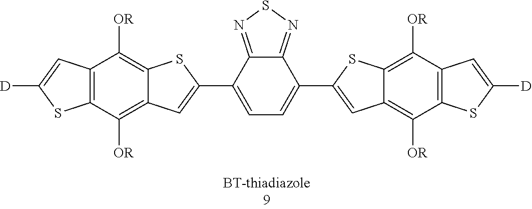

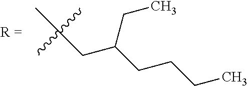 [SMILES: *CC(CC)CCCC]

| | From computational data for BT-H-thiadiazole | | | | | | From CV data for BT-thiadiazole | | |
|---|---|---|---|---|---|---|---|---|---|
| | | λ | | | | HOMO/ | | | HOMO/ |
| | | (eV, | | HOMO | LUMO | LUMO | HOMO | LUMO | LUMO |
| Compound | states | nm) | ƒ | (eV) | (eV) | gap | (eV) | (eV) | gap (eV) |
| BT-H- | S₁ | 1.78, | 1.2 | −4.52 | −2.5 | 2.02 | −4.90 | −2.64 | 2.26 |
| thiadiazole | | 696 | | | | | | | |

TABLE 4
Chemical structure and DFT/ TD-DFT calculation for BT-Py, BT-H-Py and CV data for BT-Py.

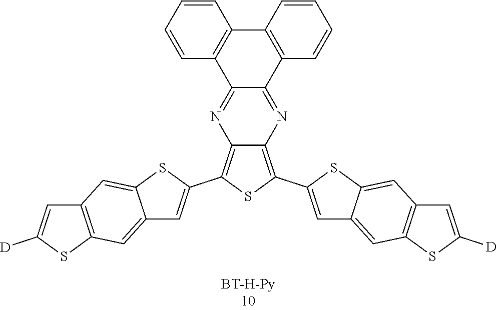 [SMILES: [2H]c1cc2cc3sc(-c4sc(-c5cc6cc7sc([2H])cc7cc6s5)c5nc6c7ccccc7c7ccccc7c6nc45)cc3cc2s1]

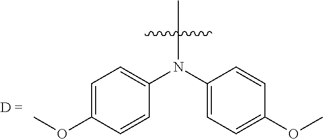

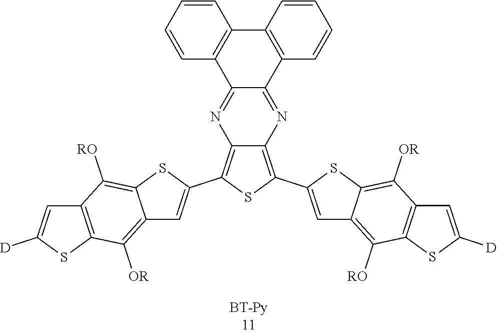

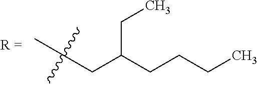 [SMILES: *CC(CC)CCCC]

| | From computational data for BT-H-Py | | | | | | From CV data for BT-Py | | |
|---|---|---|---|---|---|---|---|---|---|
| | | λ | | | | HOMO/ | | | HOMO/ |
| | | (eV, | | HOMO | LUMO | LUMO | HOMO | LUMO | LUMO gap |
| Compound | states | nm) | ƒ | (eV) | (eV) | gap | (eV) | (eV) | (eV) |
| BT-H-Py | S₁ | 1.51, | 1.51 | −4.32 | −2.69 | 1.63 | −3.38 | −4.96 | 1.58 |
| | | 821 | | | | | | | |

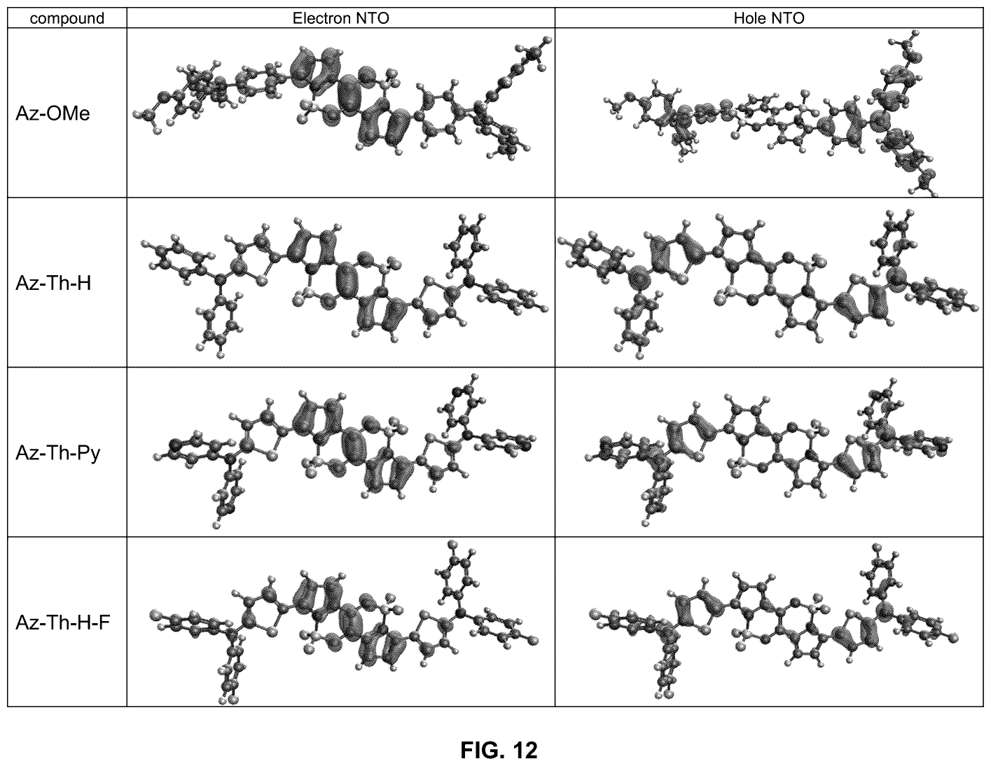

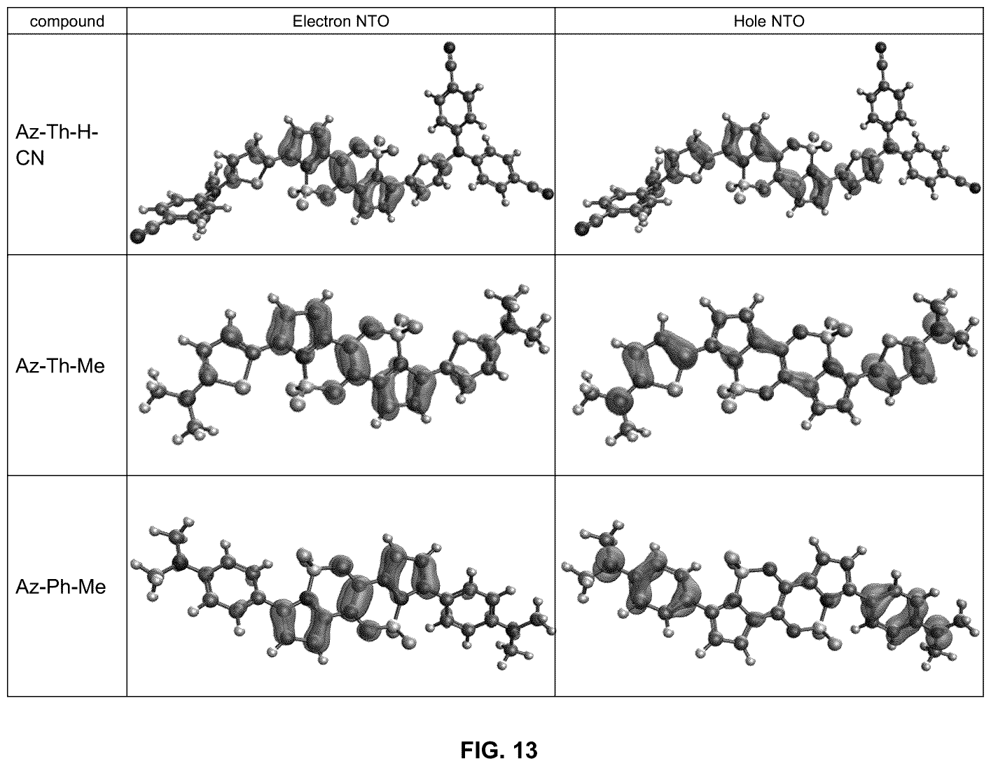

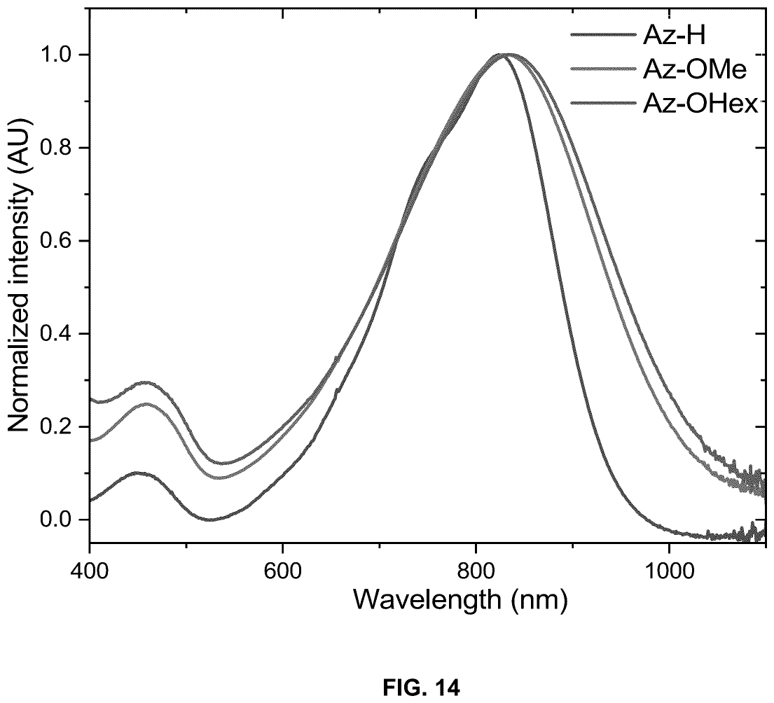

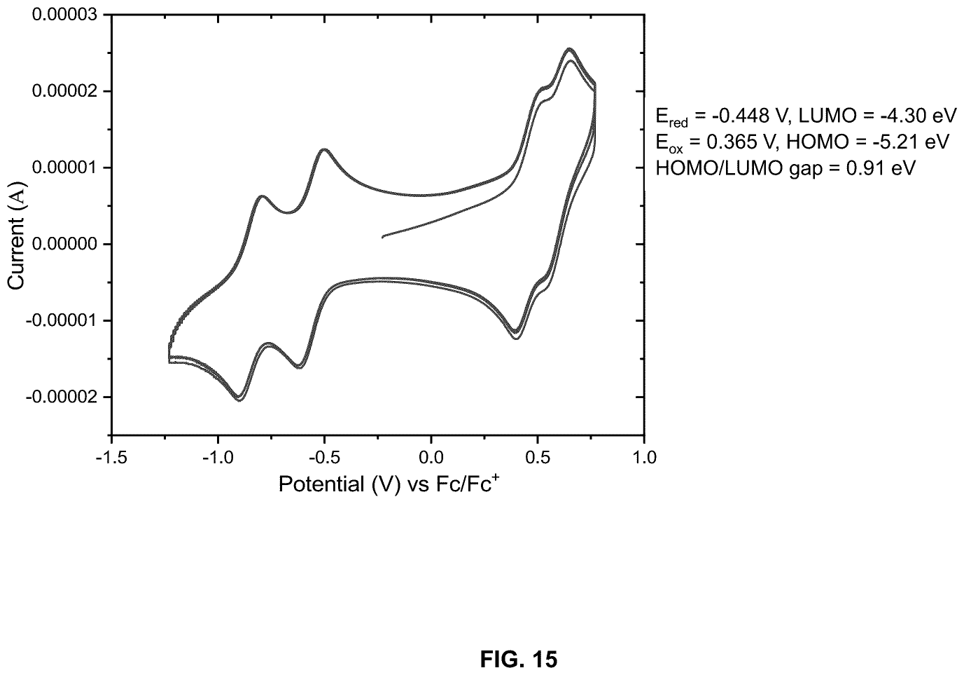

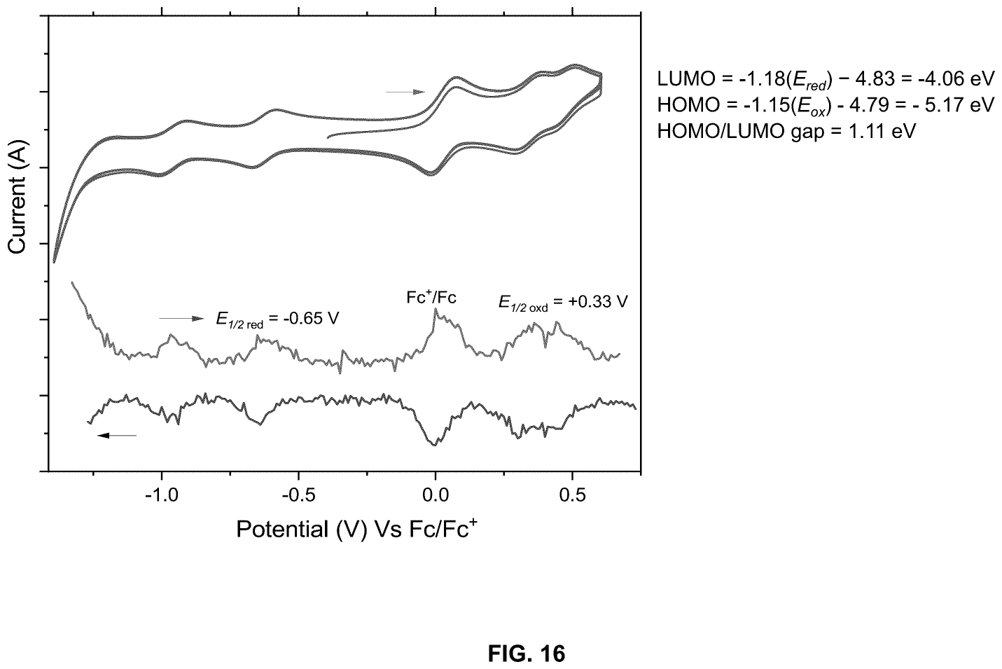

Also examined were azafulvene-type DAD structures having a central aza-fulvene core. Computational data based on these compounds are presented in Table 5. Natural transition orbitals for the aza-fulvene structures are shown in FIG. 12 and FIG. 13. UV-Vis absorption spectra of Az-H, Az-OMe, and AzOHex are presented in FIG. 14. A CV of Az-OMe is presented in FIG. 15. A CV of Az-OHex is presented in FIG. 16.

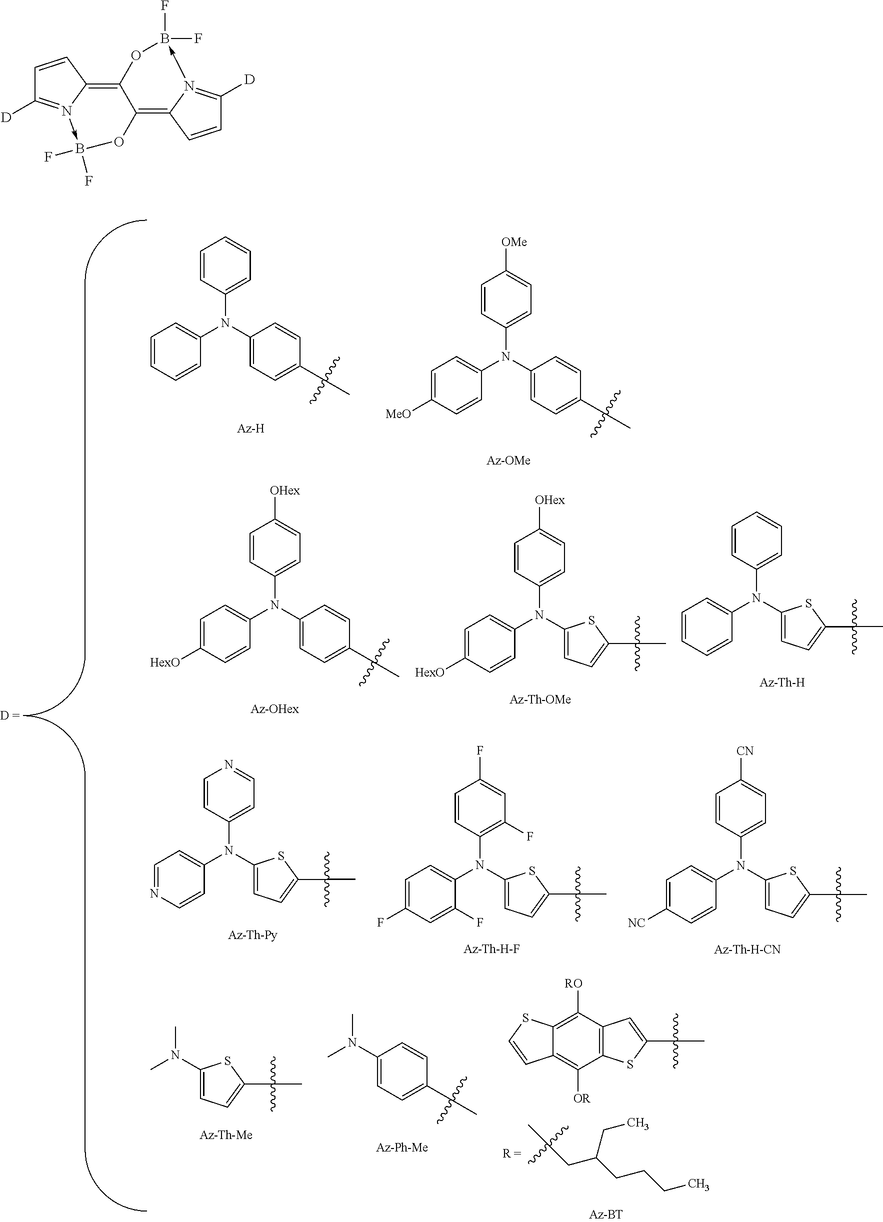

TABLE 5
Lowest energy vertical transitions, HOMO/LUMO energies
from computational and CV data for azafulvene structures.

| | From computational data for singlet states | | | | | | From CV data | | |
|---|---|---|---|---|---|---|---|---|---|
| | | λ | | | | HOMO/ | | | HOMO/ |
| | | (eV, | | HOMO | LUMO | LUMO | HOMO | LUMO | LUMO |
| compound | states | nm) | f | (eV) | (eV) | gap | (eV) | (eV) | gap |
| Az-H | S₁ | 1.56, 794 | 1.23 | −5.11 | −3.37 | 1.74 | — | — | — |
| | S₂ | 1.78, 696 | 0.004 | | | | | | |
| Az-OMe | S₁ | 1.44 | 1.23 | −4.78 | −3.18 | 1.6 | −5.21 | −4.30 | 0.91 |
| | S₂ | 1.65 | 0.015 | | | | | | |
| Az-OHex | — | — | — | — | — | — | −5.17 | −4.06 | 1.11 |
| Az-Th— | S₁ | 1.62, 765 | 1.78 | −4.54 | −2.88 | 1.66 | — | — | — |
| OMe | S₂ | 1.94, 639 | 0.001 | | | | | | |
| Az-Th—H | S₁ | 1.66, 747 | 1.71 | −4.81 | −3.10 | 1.71 | — | — | — |
| | S₂ | 1.99, 623 | 0.003 | | | | | | |
| Az-Th—Py | S₁ | 1.76, 704 | 1.46 | −5.57 | −3.70 | 1.87 | — | — | — |
| | S₂ | 2.04, 607 | 0.001 | | | | | | |
| Az-Th— | S₁ | 1.67, 744 | 1.67 | −4.95 | −3.23 | 1.72 | — | — | — |
| H—F | S₂ | 1.99, 623 | 0.003 | | | | | | |
| Az-Th—H— | S₁ | 1.69, 734 | 1.48 | −5.74 | −3.89 | 1.85 | — | — | — |
| CN | S₂ | 1.96, 632 | 0.005 | | | | | | |
| Az-Th—Me | S₁ | 1.89, 656 | 1.59 | −4.76 | −2.93 | 1.83 | — | — | — |
| | S₂ | 2.22, 558 | ~0 | | | | | | |
| Az-Ph—Me | S₁ | 1.86, 666 | 1.33 | −5.06 | −3.12 | 1.94 | — | — | — |
| | S₂ | 2.13, 582 | ~0 | | | | | | |

### Synthetic Details

### Synthesis and Characterization Data for BT-PCE

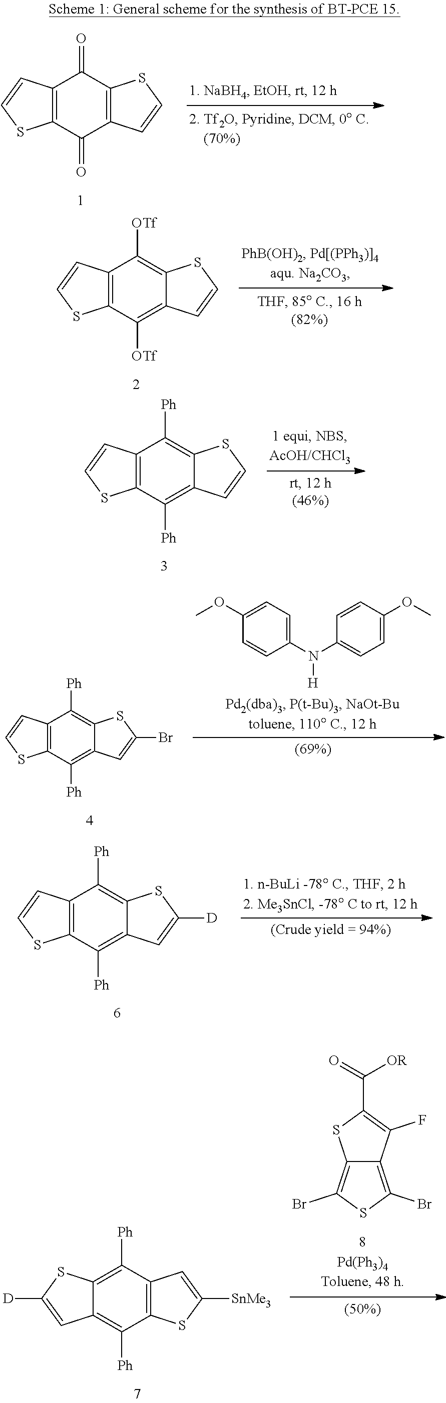

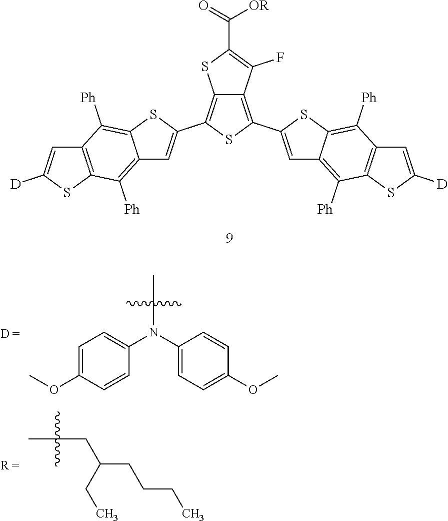

### Synthesis of Benzodithiophene-4,8-ditriflate 2

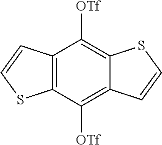 [SMILES: O=S(=O)(Oc1c2ccsc2c(OS(=O)(=O)C(F)(F)F)c2ccsc12)C(F)(F)F]

To the suspension of benzodithiophene-4,8-dione 1 (1.2 g, 5.45 mmol, 1 equiv.) in 15 mL of ethanol added sodium borohydride (453 mg, 12.0 mmol, 2.2 equiv.). It was refluxed under nitrogen for 20 h. After cooling to room temperature, the reaction mixture was poured into 10 mL of 1N HCl. Green precipitate of benzodithiophene-4,8-diol was washed several times with DI water and dried under high vacuum at 70° C. for 3 hours.

The crude material (1.1 g, 4.9 mmol, 1 equiv.) was taken as such into the next step. It was taken in a dry round bottomed flask and added 35 mL of dichloromethane under nitrogen. Then added dry pyridine (1.3 mL, 14,7 mmol, 3 equiv.) and cooled to 0° C. After dropwise addition of triflic anhydride (2.7 mL, 14,7 mmol, 3 equiv.), the reaction mixture was stirred at 0° C. for 12 h. After 12 h, reaction mixture was allowed to warm to RT. Water and 1N HCl was added. Organic layer was separated. Aqueous layer was extracted with dichloromethane (3*20 mL). Combined organic layer was washed with brine solution and dried with anhydrous sodium sulfate. Crude mixture was concentrated under reduced pressure in rotary evaporator and purified by silica gel column chromatography to isolate 1.4 g of product (Isolate yield=63%).

¹H-NMR (400 MHz, CDCl₃, δ) 7.67 (dd, J=5.6, 0.5 Hz, 2H), 7.56 (d, J=5.6 Hz, 2H).

¹³C NMR (101 MHz, CDCl₃, δ) 135.70, 132.95, 132.53, 130.39, 123.35, 120.16, 119.51, 116.97, 113.78.

### Synthesis of 4,8-diphenyl benzodithiophene 3

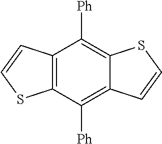 [SMILES: c1ccc(-c2c3ccsc3c(-c3ccccc3)c3ccsc23)cc1]

The triflate 2 (1.4 g, 2.9 mmol, 1 equiv.) was taken in round bottomed flask and added phenyl boronic acid (1.04 g, 8.3 mmol, 3 equiv.), tetrakis(triphenylphosphine)palladium [0] (99 mg, 0.0856 mmol, 0.05 equiv.). Then added 75 mL of dry tetrahydrofuran and previously degassed solution of 1M aqueous sodium carbonate solution. Reaction mixture was stirred under reflux for 16 h. The reaction was monitored by thin layer chromatography. After the completion of reaction, it was cooled to room temperature, quenched by adding DI water. Ethyl acetate (50 mL) was added and organic layer was separated. Aqueous layer was extracted with ethyl acetate (30 mL*3). Combined organic layer was washed with brine solution and dried with anhydrous sodium sulfate. Crude mixture was concentrated under reduced pressure in rotary evaporator and purified by silica gel column chromatography to isolate 810 mg of the product. (Isolated yield=82%).

¹H-NMR (400 MHz, CDCl₃, δ) 7.75-7.68 (m, 4H), 7.60-7.55 (m, 4H), 7.53-7.47 (m, 2H), 7.40 (d, J=5.7 Hz, 2H), 7.34 (s, 2H).

### Synthesis of 2-bromo-4,8-diphenyl benzodithiophene 4

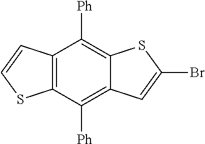 [SMILES: Brc1cc2c(-c3ccccc3)c3sccc3c(-c3ccccc3)c2s1]

4,8-diphenyl benzodithiophene 3 (1 g, 3.1 mmol, 1 equiv.), taken in a round bottomed flask. Added N-bromosuccinimide (552 mg, 3.1 mmol, 1 equiv.). Then added 50 mL of chloroform under nitrogen. This was followed by the slow addition of 8 mL of glacial acetic acid at room temperature. The reaction mixture was stirred at RT for overnight. Reaction was followed by thin layer chromatography. After 12 h of stirring, reaction mixture was quenched by adding DI water. Organic layer was separated. Aqueous layer was extracted with dichloromethane (3*10 mL). Combined organic layer was washed with saturated sodium carbonate solution and dried with anhydrous sodium sulfate. Crude mixture was concentrated under reduced pressure in rotary evaporator and purified by silica gel column chromatography to isolate 555 mg of product (Isolate yield=46%). Unreacted 4,8-diphenyl benzodithiophene (272 mg) was recovered. Small amount of dibromo product also formed.

¹H-NMR (400 MHz, CDCl₃, δ) δ 7.67 (ddd, J=8.1, 2.3, 1.3 Hz, 4H), 7.60-7.54 (m, 4H), 7.53-7.47 (m, 2H), 7.43 (d, J=5.7 Hz, 1H), 7.33-7.28 (m, 2H).

¹³C NMR (101 MHz, CDCl₃, δ) 139.03, 138.78, 138.71, 138.48, 136.00, 135.65, 129.72, 129.67, 129.26, 129.13, 128.99, 128.96, 128.45, 128.41, 127.60, 125.61, 122.88, 116.12.

### Synthesis of 2-(N,N-diphenylamine)-4,8-diphenyl benzodithiophene 6

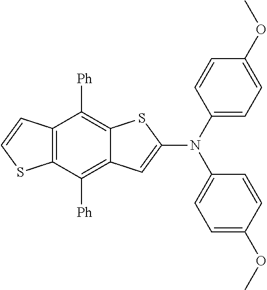 [SMILES: COc1ccc(N(c2ccc(OC)cc2)c2cc3c(-c4ccccc4)c4sccc4c(-c4ccccc4)c3s2)cc1]

2-bromo-4,8-diphenyl benzodithiophene 4 (555 mg, 1.31 mmol, 1 equiv.) taken in in a round bottomed flask. Added N,N-4-methoxydiphenylamine (308 mg, 1.34 mmol, 1.02 equiv), tris(dibenzylideneacetone)dipallad-ium[0] (24 mg, 0.02 mmol, 0.015 equiv.), tri-tert-butylphosphine (0.2 mL of 10% W/W solution, 0.06 mmol, 0.046 equiv.) and sodium tert-butoxide (132 mg, 1.37 mmol, 1.04 equiv.). Then added 50 mL of dry toluene. The reaction mixture was stirred under reflux for 24 h. Reaction was monitored by thin layer chromatography. After the completion of reaction, it was cooled to RT and DI water was added. Organic layer was separated. Aqueous layer was extracted with ethyl acetate (3*20 mL). Combined organic layer was washed with brine solution and dried with anhydrous sodium sulfate. Crude mixture was concentrated under reduced pressure in rotary evaporator and purified by silica gel column chromatography to isolate 519 mg of product (Isolated yield=69%).

¹H-NMR (400 MHz, CDCl₃, δ) 7.67-7.61 (m, 4H), 7.49 (tdd, J=8.1, 7.4, 2.9, 1.8 Hz, 4H), 7.44-7.37 (m, 2H), 7.25-7.19 (m, 2H), 7.15-7.10 (m, 4H), 6.82-6.76 (m, 4H), 6.54 (s, 1H), 3.77 (s, 6H).

### Synthesis of trimethyl tin Derivative 7

2-(N,N-diphenylamine)-4,8-diphenyl benzodithiophene 7 (430 mg, 0.7547 mmol, 1.0 equiv.) was taken in a dry round bottomed flask. Added 25 mL of dry THF. Reaction mixture was cooled to −78° C. Then slowly added n-BuLi (2.5 M, 0.326 mL, 0.839 mmol, 1.1 equiv.) and stirred for 2 h at the same temperature. After 2 h, the reaction mixture was allowed warm to 0° C. and stirred for 15 min. Then it was further cooled to −78° C. Added trimethyl tin chloride (1 M, 1 mL, 1.0 mmol, 1.3 equiv.). It was allowed to stir at the same temperature for 4 h. Then temperature was slowly allowed to warm to RT and further stirred at RT for 12 h. Then it was quenched by adding water and 10 mL of ethyl acetate was added. Organic layer was separated. Aqueous layer was extracted with ethyl acetate (3*10 mL). Combined organic layer was washed with brine solution and dried with anhydrous sodium sulfate. The mixture was concentrated under reduced pressure to get 516 mg of the material. It was taken as such to the next step without further purification. Product was confirmed from the NMR of crude material.

### Synthesis of BT-PCE 9

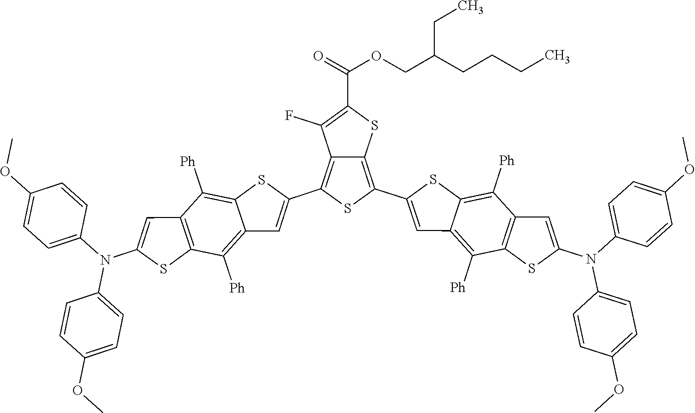 [SMILES: CCCCC(CC)COC(=O)c1sc2c(-c3cc4c(-c5ccccc5)c5sc(N(c6ccc(OC)cc6)c6ccc(OC)cc6)cc5c(-c5ccccc5)c4s3)sc(-c3cc4c(-c5ccccc5)c5sc(N(c6ccc(OC)cc6)c6ccc(OC)cc6)cc5c(-c5ccccc5)c4s3)c2c1F]

Trimethyl tin derivative 7 was taken in a round bottomed flask (500 mg, 0.557 mmol, 2.0 equiv.). Added 2-ethylhexyl-4,6-dibromo-3-fluorothieno [3,4-b] thiophene-2-carboxylate 8 (100 mg, 0.2785 mmol, 1.0 equiv.) and tetrakis(triphenylphosphine)palladium [0] (30 mg, 0.0259 mmol, 0.01 equiv.). Then added 20 mL of dry toluene. The reaction mixture was refluxed for 48 h under nitrogen. Reaction was monitored by thin layer chromatography. After the completion of reaction, toluene was evaporated under reduced pressure. Crude mixture was purified by silica gel column chromatography to isolate 130 mg of product (Isolated yield=47%).

¹H-NMR (400 MHz, CDCl₃, δ) δ 7.74-7.35 (m, 22H), 7.18-7.05 (m, 8H), 6.80 (d, J=8.4 Hz, 8H), 6.49-6.44 (m, 2H), 4.23 (t, J=5.6 Hz, 1H), 3.78 (s, 13H), 1.46 (q, J=7.2 Hz, 2H), 1.42-1.27 (m, 7H), 0.93 (dt, J=20.2, 7.2 Hz, 6H).

### Synthesis of BT-thiadiazole:

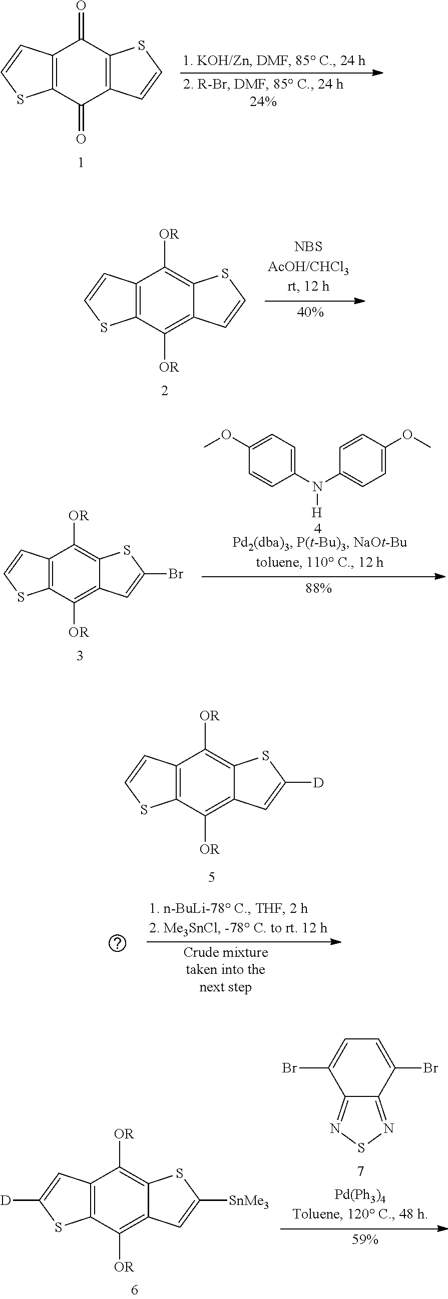

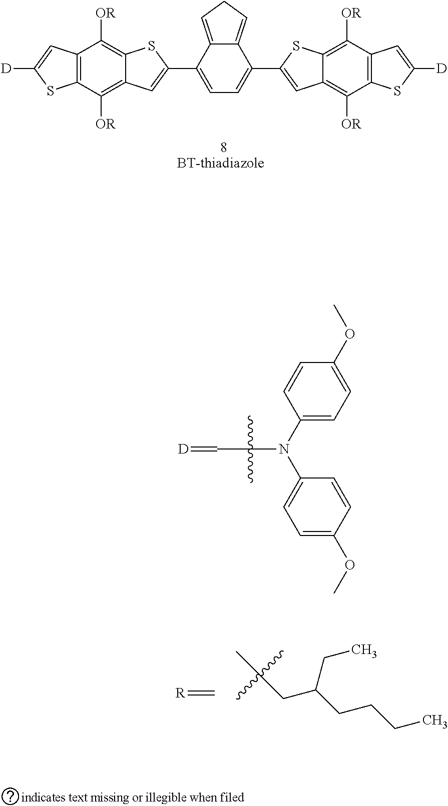

### Synthesis of 4,8-(2-ethyl-1-hexyloxy) benzodithiophene 2

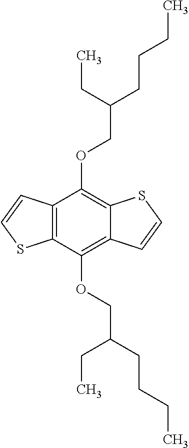 [SMILES: CCCCC(CC)COc1c2ccsc2c(OCC(CC)CCCC)c2ccsc12]

To the suspension of benzodithiophene-4,8-dione (2 g, 9.08 mmol, 1.0 equiv.) in DMF added KOH pellets (10.1 g, 181.6 mmol, 20 equiv.) and zinc (10.1 g, 154.3 mmol, 17 equiv.) and stirred at 85° C. for 24 h. After 24 h, reaction mixture was cooled to RT, added 1-bromo-2-ethylhexane and further stirred for 24 h at 85° C. Reaction mixture was cooled to RT, filtered. Filtrate was concentrated under the reduced pressure. Crude mixture was purified on silica gel column chromatography to isolate 843 mg of product (Isolated yield=21%).

¹H-NMR (400 MHz, Acetone-d₆, δ) 7.64 (d, J=5.5 Hz, 2H), 7.55 (d, J=5.6 Hz, 2H), 4.23 (d, J=5.3 Hz, 4H), 1.86-1.33 (m, 19H), 1.03 (t, J=7.4 Hz, 6H), 0.98-0.90 (m, 6H).

¹³C-NMR (101 MHz, Acetone-d₆, δ) 145.48, 132.27, 130.93, 127.67, 120.98, 76.63, 41.59, 31.26, 29.97, 24.62, 23.77, 14.40, 11.66.

### Synthesis of 2-bromo-4,8-(2-ethyl-1-hexyloxy) benzodithiophene 3

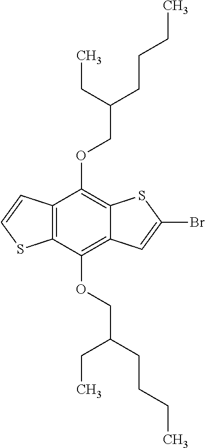 [SMILES: CCCCC(CC)COc1c2cc(Br)sc2c(OCC(CC)CCCC)c2ccsc12]

4,8-(2-ethyl-1-hexyloxy) benzodithiophene 2 (200 mg, 0.4477 mmol, 1 equiv.), taken in a round bottomed flask. Added N-bromosuccinimide (80 mg, 0.4494 mmol, 1 equiv.). Then added 5 mL of dry dichloromethane under nitrogen. This was followed by the slow addition of 0.8 mL of glacial acetic acid at room temperature. The reaction mixture was stirred at RT for overnight. Reaction was followed by thin layer chromatography. After 12 h of stirring, reaction mixture was quenched by adding DI water. Organic layer was separated. Aqueous layer was extracted with dichloromethane (3*10 mL). Combined organic layer was washed with saturated sodium bicarbonate solution and dried with anhydrous sodium sulfate. Crude mixture was concentrated under reduced pressure in rotary evaporator and purified by silica gel column chromatography to isolate 107 mg of product (Isolate yield=40%).

1H NMR (400 MHz, Acetone-d₆, δ) 7.71 (d, J=5.6 Hz, 1H), 7.62 (s, 1H), 7.56 (d, J=5.6 Hz, 1H), 4.23 (t, J=5.4 Hz, 4H), 1.87-1.32 (m, 19H), 1.02 (t, J=7.5 Hz, 6H), 0.94 (t, J=7.1 Hz, 6H).

### Synthesis of 2-(N,N-diphenylamine)-4,8-di(2-ethyl-1-hexyloxy) benzodithiophene 5

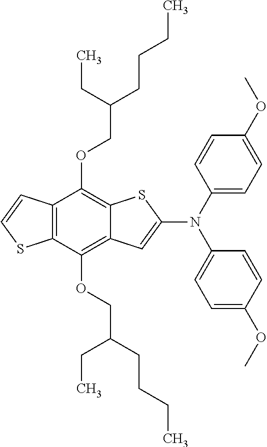 [SMILES: CCCCC(CC)COc1c2cc(N(c3ccc(OC)cc3)c3ccc(OC)cc3)sc2c(OCC(CC)CCCC)c2ccsc12]

2-bromo-4,8- di(2-ethyl-1-hexyloxy) benzodithiophene 5 (93 mg, 0.1769 mmol, 1.0 equiv.) taken in a round bottomed flask. Added N,N-4-methoxydiphenylamine (48 mg, 0.2123 mmol, 1.2 equiv.), tris(dibenzylideneacetone)dipalladium[0] (3 mg, 0.0035 mmol, 0.02 equiv.), tri-tert-butylphosphine (0.02 mL of 10% W/W solution, 0.007 mmol, 0.04 equiv.) and sodium tert-butoxide (20 mg, 0.2123 mmol, 1.2 equiv.). Then added 5 mL of dry toluene. The reaction mixture was stirred under reflux for 24 h. Reaction was monitored by thin layer chromatography. After the completion of reaction, it was cooled to RT and DI water was added. Organic layer was separated. Aqueous layer was extracted with ethyl acetate (3*5 mL). Combined organic layer was washed with brine solution and dried with anhydrous sodium sulfate. Crude mixture was concentrated under reduced pressure in rotary evaporator and purified by silica gel column chromatography to isolate 105 mg of product (Isolated yield=88%).

¹H-NMR (400 MHz, Acetone-d₆, δ) 7.48-7.41 (m, 2H), 7.30-7.25 (m, 4H), 6.99-6.94 (m, 4H), 6.43 (s, 1H), 4.05 (dd, J=38.7, 5.1 Hz, 4H), 3.81 (s, 6H), 1.72-1.24 (m, 18H), 0.98-0.81 (m, 12H).

### Synthesis of trimethyl tin Derivative 6

2-(N,N-diphenylamine)-4,8-diphenyl benzodithiophene 5 (100 mg, 0.1567 mmol, 1.0 equiv.) was taken in a dry round bottomed flask. Added 55 mL of dry THF. Reaction mixture was cooled to −78° C. Then slowly added n-BuLi (2.5 M, 0.326 mL, 0.839 mmol, 1.1 equiv.) and stirred for 2 h at the same temperature. After 2 h, the reaction mixture was allowed warm to 0° C. and stirred for 15 min. Then it was further cooled to −78° C. Added trimethyl tin chloride (1 M, 0.203 mL, 0.2037 mmol, 1.3 equiv.). It was allowed to stir at the same temperature for 4 h. Then temperature was slowly allowed to warm to RT and was further stirred at RT for 12 h. Then it was quenched by adding water. Added 10 mL of ethyl acetate. Organic layer was separated. Aqueous layer was extracted with ethyl acetate (3*10 mL). Combined organic layer was washed with brine solution and dried with anhydrous sodium sulfate. The mixture was concentrated under reduced pressure to get crude material. Product was confirmed from the NMR of crude material. It was taken as such to the next step without further purification.

### Synthesis of BT-thiadiazole 8

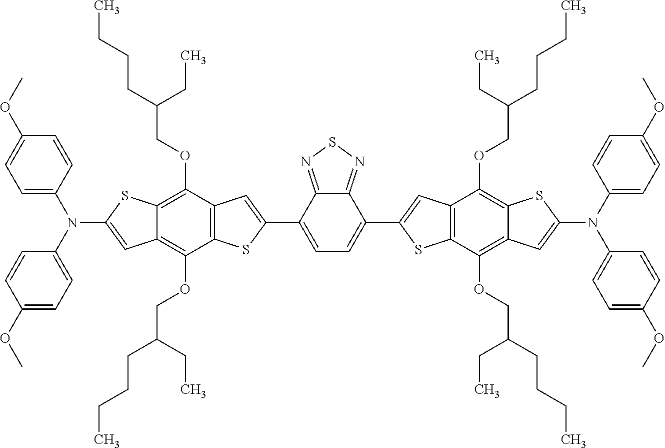 [SMILES: CCCCC(CC)COc1c2cc(N(c3ccc(OC)cc3)c3ccc(OC)cc3)sc2c(OCC(CC)CCCC)c2cc(-c3ccc(-c4cc5c(OCC(CC)CCCC)c6sc(N(c7ccc(OC)cc7)c7ccc(OC)cc7)cc6c(OCC(CC)CCCC)c5s4)c4nsnc34)sc12]

Trimethyl tin derivative 6 was taken in a round bottomed flask (124 mg, 0.1502 mmol, 2.1 equiv.). Added 4,7-Dibromobenzothiadiazole (21 mg, 0.0714 mmol, 1.0 equiv.) and tetrakis(triphenylphosphine)palladium [0] (8 mg, 0.0071 mmol, 0.01 equiv.). Then added 5 mL of dry toluene. The reaction mixture was refluxed for 48 h under nitrogen. Reaction was monitored by thin layer chromatography. After the completion of reaction, toluene was evaporated under reduced pressure. Crude mixture was purified by silica gel column chromatography to isolate 65 mg of the product (Isolated yield=59%).

¹H-NMR (600 MHz, CDCl₃, δ) 8.79 (s, 2H), 7.91 (s, 2H), 7.25 (d, J=6.5 Hz, 8H), 6.92-6.84 (m, 8H), 6.54 (s, 2H), 4.13 (dd, J=79.1, 5.4 Hz, 8H), 3.83 (s, 12H), 1.80 (p, J=6.0 Hz, 2H), 1.76-1.64 (m, 6H), 1.46-1.26 (m, 23H), 1.03 (t, J=7.4 Hz, 7H), 0.98-0.87 (m, 19H).

¹³C-NMR (151 MHz, CDCl₃, δ) 156.80, 140.29, 126.22, 114.63, 76.12, 55.49, 40.65, 40.51, 30.52, 30.43, 29.22, 29.13, 23.86, 23.78, 23.17, 23.07, 14.17, 14.13, 11.38, 11.2

### Synthetic Details for azafulvene Structures

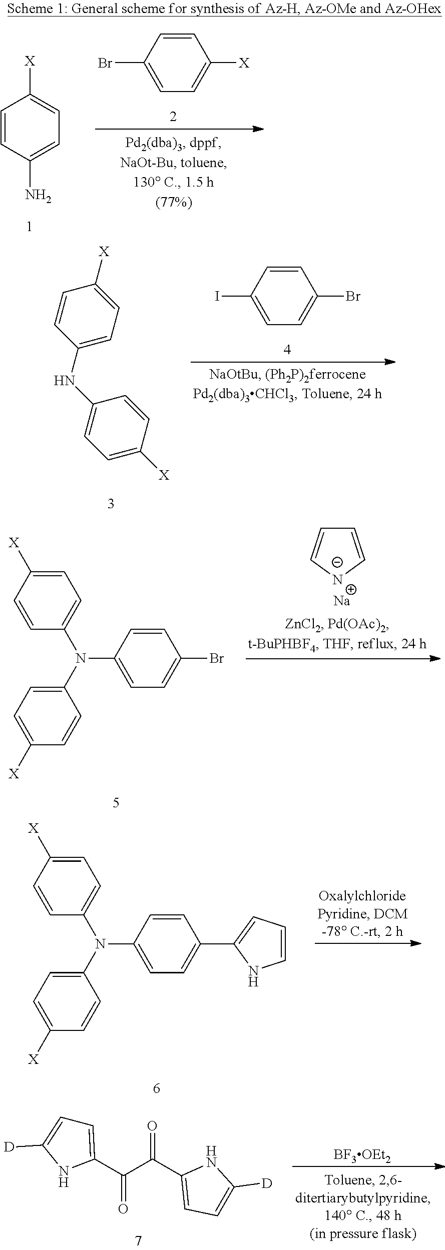

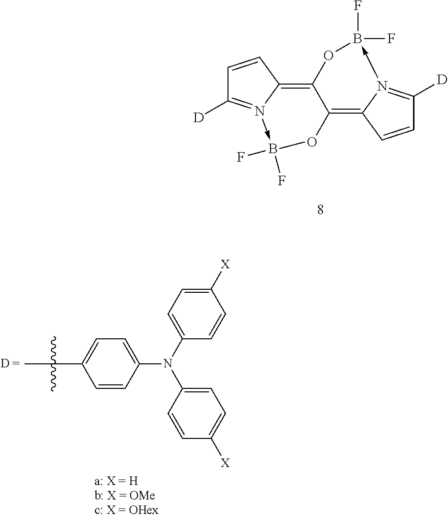

### Synthesis of N,N-4-methoxydipehnyl amine 3b

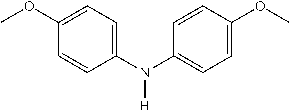

In a clean round bottomed flask, p-anisidine (10 g, 81.2 mmol, 1 equiv.), tris(dibenzylideneacetone)dipalladium[0] (372 mg, 0.406 mmol, 0.5 mol%) and 1,1′-bis(diphenylphosphino)ferrocene (450 mg, 0.812 mmol, 1 mol %). Added sodium tert-butoxide (7.8 g, 81.2 mmol, 1.0 equiv.). To this mixture dry toluene (200 mL) was added. Then 1-bromo-4-methoxy benzene was added. The mixture was stirred under reflux condition for 1.5 h. Reaction was monitored by thin layer chromatography. After the reaction, it was quenched by adding DI water. Organic layer was separated. Aqueous layer was extracted with ethyl acetate (3*50 mL). Combined organic layer was washed with brine solution and dried with anhydrous sodium sulfate. Crude mixture was concentrated under reduced pressure in rotary evaporator. It was further dissolved in minimal amount of dichloromethane. The product was crashed out by adding hexanes and filtered to isolate 15.4 g of product 7 (Yield =83%).

¹H-NMR (400 MHz, CDCl₃, δ) 6.98-6.89 (m, 4H), 6.85-6.79 (m, 4H), 5.27 (s, 1H), 3.78 (s, 6H).

¹³C-NMR (101 MHz, CDCl₃) δ 153.45, 137.13, 118.73, 113.90, 54.82.

### Synthesis of N,N-4-hexyloxydipehnyl amine 3c

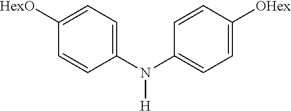

In a clean round bottomed flask, p-(hexyloxy)aniline (4 g, 19.3 mmol, 1 equiv.), tris(dibenzylideneacetone) dipalladium[0] (75 mg, 0.0819 mmol, 0.4 mol %) and 1,1′-bis(diphenylphosphino)ferrocene (282 mg, 0.509 mmol, 2.6 mol %). Added sodium tert-butoxide (1.95 g, 20.2 mmol, 1.05 equiv.). To this mixture dry toluene (200 mL) was added. Then 1-bromo-4-hexyloxy benzene was added. The mixture was stirred under reflux condition for 1.5 h. Reaction was monitored by thin layer chromatography. After the reaction, it was quenched by adding DI water. Organic layer was separated. Aqueous layer was extracted with ethyl acetate (3*30 mL). Combined organic layer was washed with brine solution and dried with anhydrous sodium sulfate. Crude mixture was concentrated under reduced pressure in rotary evaporator. It was further dissolved in minimal amount of dichloromethane. The product was crashed out by adding hexanes and filtered to isolate 4.6 g of product 7 (Yield=61%).

1H-NMR (400 MHz, Acetone-d₆, δ) 6.98-6.93 (m, 4H), 6.84-6.78 (m, 4H), 6.72 (s, 1H), 3.92 (t, J=6.5 Hz, 4H), 1.78-1.68 (m, 4H), 1.51-1.42 (m, 4H), 1.41-1.28 (m, 8H), 0.97-0.82 (m, 6H).

¹³C NMR (101 MHz, Acetone-d₆) δ 154.19, 139.31, 119.55, 116.12, 68.90, 32.38, 30.17, 26.52, 23.31, 14.31.

### Synthesis of N,N-bis(4-methoxypehnyl)-4-bromoaniline 5b

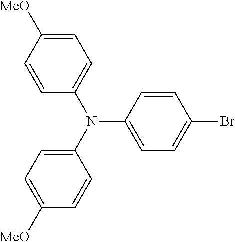 [SMILES: COc1ccc(N(c2ccc(Br)cc2)c2ccc(OC)cc2)cc1]

N,N-4-methoxydiphenylamine 3b (6 g, 26.16 mmol, 1.0 equiv.) taken in a round bottomed flask. Added 1-bromo-4-iodobenzene (8.8 g, 31.4 mmol, 1.2 equiv.), tris(dibenzylideneacetone)dipalladium[0] (256 mg, 0.2473 mmol, 0.01 equiv.), 1,1′-Bis(diphenylphosphino)ferrocene (273 mg, 0.494 mmol, 0.02 equiv.) and sodium tert-butoxide (3.8 g, 39.26 mmol, 1.5 equiv.). Then added 40 mL of dry toluene. The reaction mixture was stirred under reflux for 24 h. Reaction was monitored by thin layer chromatography. After the completion of reaction, it was cooled to RT and DI water was added. Organic layer was separated. Aqueous layer was extracted with ethyl acetate (3*20 mL). Combined organic layer was washed with brine solution and dried with anhydrous sodium sulfate. Crude mixture was concentrated under reduced pressure in rotary evaporator and purified by silica gel column chromatography to isolate 8.2 g of product (Isolated yield=82%).

¹H-NMR (400 MHz, CDCl₃, δ) 7.24 (d, J=8.5 Hz, 2H), 7.03 (d, J=8.0 Hz, 4H), 6.89-6.71 (m, 6H), 3.79 (s, 6H).

Synthesis of N,N-bis(4-hexyloxypehnyl)-4-bromoaniline 5c

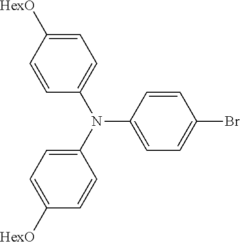

N,N-4-methoxydiphenylamine 3c (2 g, 5.03 mmol, 1.0 equiv.) taken in a round bottomed flask. Added 1-bromo-4-iodobenzene (1.57 g, 5.53 mmol, 1.1 equiv.), tris(dibenzylideneacetone)dipalladium [0] (46 mg, 0.050 mmol, 0.01 equiv.), 1,1′-Bis(diphenylphosphino)ferrocene (56 mg, 0.100 mmol, 0.02 equiv.) and sodium tert-butoxide (0.725 mg, 7.55 mmol, 1.5 equiv.). Then added 25 mL of dry toluene. The reaction mixture was stirred under reflux for 24 h. Reaction was monitored by thin layer chromatography. After the completion of reaction, it was cooled to RT and DI water was added. Organic layer was separated. Aqueous layer was extracted with ethyl acetate (3*20 mL). Combined organic layer was washed with brine solution and dried with anhydrous sodium sulfate. Crude mixture was concentrated under reduced pressure in rotary evaporator and purified by silica gel column chromatography to isolate 2.4 g of product (Isolated yield=86%).

¹H NMR (400 MHz, Acetone-d₆, δ) 7.33-7.24 (m, 2H), 7.10-6.99 (m, 4H), 6.95-6.86 (m, 4H), 6.78-6.71 (m, 2H), 3.98 (t, J=6.5 Hz, 4H), 1.81-1.71 (m, 4H), 1.53-1.41 (m, 4H), 1.41-1.25 (m, 8H), 0.99-0.82 (m, 6H).

### Synthesis of 1-(N,N-diphenyl)-4-1H-pyrrole benzene 6a

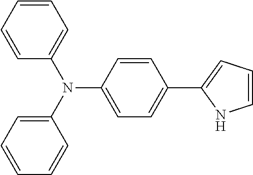 [SMILES: c1ccc(N(c2ccccc2)c2ccc(-c3ccc[nH]3)cc2)cc1]

Anhydrous zinc chloride (2.53 g, 18.5 mmol, 3.0 equiv) was taken in a dry round bottomed Schlenk flask and added 25 mL of dry THF under nitrogen. In another dry Schlenk flask, sodium hydride (444 mg, 18.5 mmol, 3.0 equiv.) weighed and added 12 mL of dry THF under nitrogen. The mixture was cooled to 0° C. and added pyrrole (2 mL, 18.5 mmol, 3 equiv.) followed by the stirring for 15 min. to form sodium pyrrolide. After 15 min. zinc chloride solution was cannula transferred to the sodium pyrrolide solution and stirred for additional 15 min. Then added 4 bromotriphenylamine (2 g, 6.17 mmol, 1 equiv.) palladium acetate (55 mg, 0.2467 mmol, 0.04 equiv.) and tri-tent-butylphosphonium tetrafluoroborate (143 mg, 0.4935 mmol, 0.08 equiv.) under nitrogen. The reaction mixture was refluxed for 20 h. Reaction was monitored by thin layer chromatography. After the completion of reaction, it was cooled to RT. DI water and 20 mL of ethyl acetate was added. Undissolved particles were filtered through Celite. Organic layer was separated. Aqueous layer was extracted with ethyl acetate (3*20 mL). Combined organic layer was washed with brine solution and dried with anhydrous sodium sulfate. Crude mixture was concentrated under reduced pressure in rotary evaporator and purified by silica gel column chromatography to isolate 1.9 g of product (Isolated yield=52%).

¹H-NMR (400 MHz, Acetone-d₆, δ) 10.42 (s, 1H), 7.56 (ddt, J=8.4, 4.7, 1.9 Hz, 2H), 7.34-7.21 (m, 4H), 7.10-6.97 (m, 8H), 6.83 (ddq, J=5.7, 2.5, 1.4 Hz, 1H), 6.47 (dddt, J=4.1, 3.3, 2.5, 1.3 Hz, 1H), 6.18-6.12 (m, 1H).

¹³C-NMR (101 MHz, Acetone-d₆, δ) 147.84, 145.40, 131.44, 129.29, 128.54, 124.59, 124.50, 123.76, 122.66, 118.67, 109.24, 105.09.

### Synthesis of 1-(N,N-4-methoxy diphenyl)-4-1H-pyrrole benzene 6b

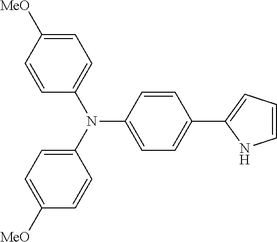 [SMILES: COc1ccc(N(c2ccc(OC)cc2)c2ccc(-c3ccc[nH]3)cc2)cc1]

Anhydrous zinc chloride (2.13 g, 15.61 mmol, 3.0 equiv) was taken in a dry round bottomed Schlenk flask and added 25 mL of dry THF under nitrogen. In another dry Schlenk flask, sodium hydride (374 mg, 15.61 mmol, 3.0 equiv.) weighed and added 12 mL of dry THF under nitrogen. The mixture was cooled to 0° C. and added pyrrole (1.08 mL, 15.61 mmol, 3 equiv.) followed by the stirring for 15 min. to form sodium pyrrolide. After 15 min. zinc chloride solution was cannula transferred to the sodium pyrrolide solution and stirred for additional 15 min. Then added N,N-bis(4-methoxypehnyl)-4-bromoaniline (2 g, 5.2 mmol, 1 equiv.) palladium acetate (47 mg, 0.2081 mmol, 0.04 equiv.) and tri-tert-butylphosphonium tetrafluoroborate (73 mg, 0.4161 mmol, 0.08 equiv.) under nitrogen. The reaction mixture was refluxed for 20 h. Reaction was monitored by thin layer chromatography. After the completion of reaction, it was cooled to RT. DI water and 20 mL of ethyl acetate was added. Undissolved particles were filtered through Celite. Organic layer was separated. Aqueous layer was extracted with ethyl acetate (3*20 mL). Combined organic layer was washed with brine solution and dried with anhydrous sodium sulfate. Crude mixture was concentrated under reduced pressure in rotary evaporator and purified by silica gel column chromatography to isolate 1.5 g of product (Isolated yield=72%).

¹H NMR (500 MHz, Chloroform-d, δ) 8.32 (s, 1H), 7.28 (dd, J=7.6, 5.6 Hz, 2H), 7.05 (dd, J=9.0, 2.1 Hz, 4H), 6.97-6.91 (m, 2H), 6.82 (dd, J=7.6, 5.4 Hz, 5H), 6.40 (d, J=3.7 Hz, 1H), 6.27 (q, J=2.9 Hz, 1H), 3.80 (s, 6H).

¹³C NMR

### Synthesis of 1-(N,N-4-hexyloxy diphenyl)-4-1H-pyrrole benzene 6c.

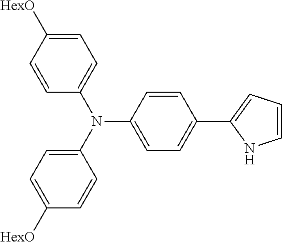

Anhydrous zinc chloride (1.78 g, 13.03 mmol, 3.0 equiv) was taken in a dry round bottomed Schlenk flask and added 25 mL of dry THF under nitrogen. In another dry Schlenk flask, sodium hydride (0.3127 mg, 13.03 mmol, 3.0 equiv.) weighed and added 15 mL of dry THF under nitrogen. The mixture was cooled to 0° C. and added pyrrole (0.904 mL, 13.03 mmol, 3 equiv.) followed by the stirring for 15 min. to form sodium pyrrolide. After 15 min. zinc chloride solution was cannula transferred to the sodium pyrrolide solution and stirred for additional 15 min. Then canula transferred solution of N,N-bis(4-hexyloxypehnyl)-4-bromoaniline in 15 mL dry THF, (2.4 g, 4.34 mmol, 1 equiv.) and added palladium acetate (47 mg, 0.2081 mmol, 0.04 equiv.) and tri-tent-butylphosphonium tetrafluoroborate (73 mg, 0.4161 mmol, 0.08 equiv.) under nitrogen. The reaction mixture was refluxed for 20 h. Reaction was monitored by thin layer chromatography. After the completion of reaction, it was cooled to RT. DI water and 20 mL of ethyl acetate was added. Undissolved particles were filtered through Celite. Organic layer was separated. Aqueous layer was extracted with ethyl acetate (3*20 mL). Combined organic layer was washed with brine solution and dried with anhydrous sodium sulfate. Crude mixture was concentrated under reduced pressure in rotary evaporator and purified by silica gel column chromatography to isolate 2.28 g of product (Isolated yield=67%).

¹H-NMR (400 MHz, Acetone-d₆, δ) δ 10.32 (s, 1H), 7.48-7.43 (m, 2H), 7.04-6.97 (m, 4H), 6.91-6.84 (m, 6H), 6.79 (td, J=2.7, 1.5 Hz, 1H), 6.38 (ddd, J=3.5, 2.6, 1.5 Hz, 1H), 6.12 (ddd, J=3.5, 2.7, 2.3 Hz, 1H), 3.97 (s, 4H), 1.82-1.71 (m, 4H), 1.54-1.41 (m, 4H), 1.36 (dddd, J=8.0, 6.9, 3.9, 2.4 Hz, 8H), 0.95-0.86 (m, 6H).

¹³C-NMR (101 MHz, Acetone-d₆, δ) 155.41, 146.64, 140.82, 131.72, 126.26, 126.08, 124.20, 121.03, 118.08, 115.16, 108.99, 104.30, 67.79, 31.36, 25.51, 22.30, 13.31.

### Synthesis of Diketo Precursor 7a

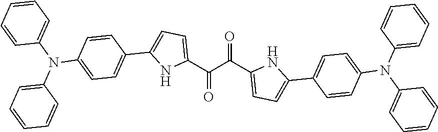 [SMILES: O=C(C(=O)c1ccc(-c2ccc(N(c3ccccc3)c3ccccc3)cc2)[nH]1)c1ccc(-c2ccc(N(c3ccccc3)c3ccccc3)cc2)[nH]1]

In a dry round bottomed Schlenk flask, added 15 mL of dry dichloromethane under nitrogen. Then added oxalyl chloride (0.14 mL, 1.6 mmol, 1.0 equiv.) and the reaction mixture was cooled to −78° C. To this mixture dry pyridine was added (0.26 mL, 3.2 mmol, 2.0 equiv.) and further stirred for 40 min. at the same temperature. Then cannula transferred the solution of 1-(N,N-diphenyl)-4-1H-pyrrole benzene 4a (1 g, 3.2 mmol, 2.0 equiv.) in 15 mL of dry dichloromethane.

The reaction mixture was further stirred for 15 minutes at the same temperature. The mixture was allowed to warm to RT. The reaction mixture was quenched by adding water. Organic layer was separated. Aqueous layer was extracted with dichlormethane (3*20 mL). Combined organic layer wad washed with brine solution and dried with anhydrous sodium sulfate. The crude mixture was concentrated under reduced pressure in rotary evaporator and purified by basic alumina column chromatography to isolate 546 mg of the product (Isolated yield=51%).

¹H-NMR (400 MHz, Acetone-d₆, δ) 11.21 (s, 2H), 7.86 (d, J=8.7 Hz, 4H), 7.37-7.33 (m, 8H), 7.17-7.03 (m, 20H), 6.70 (dd, J=4.1, 2.5 Hz, 2H).

### Synthesis of Diketo Precursor 7b

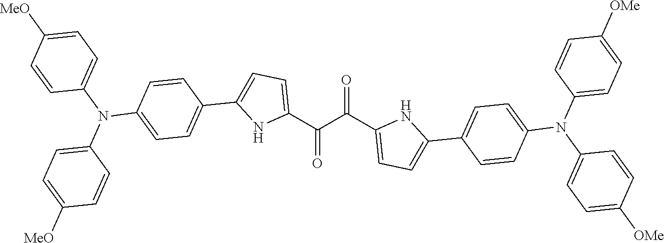 [SMILES: COc1ccc(N(c2ccc(OC)cc2)c2ccc(-c3ccc(C(=O)C(=O)c4ccc(-c5ccc(N(c6ccc(OC)cc6)c6ccc(OC)cc6)cc5)[nH]4)[nH]3)cc2)cc1]

In a dry round bottomed Schlenk flask, added 15 mL of dry dichloromethane under nitrogen. Then added oxalyl chloride (0.058 mL, 0.67 mmol, 1.0 equiv.) and the reaction mixture was cooled to −78° C. To this mixture dry pyridine was added (0.11 mL, 1.35 mmol, 2.0 equiv.) and further stirred for 40 min. at the same temperature. Then cannula transferred the solution of 1-bis(N,N-4-methoxyphenyl)-4-1H-pyrrole benzene 4b (0.5 g, 1.35 mmol, 2.0 equiv.) in 15 mL of dry dichloromethane. The reaction mixture was further stirred for 15 minutes at the same temperature. The mixture was allowed to warm to RT. The reaction mixture was quenched by adding water. Organic layer was separated. Aqueous layer was extracted with dichlormethane (3*20 mL). Combined organic layer wad washed with brine solution and dried with anhydrous sodium sulfate. The crude mixture was concentrated under reduced pressure in rotary evaporator and purified by basic alumina column chromatography to isolate 300 mg of the product (Isolated yield=56%).

¹H-NMR (600 MHz, Acetone-d₆, δ) 11.12 (s, 2H), 7.80-7.72 (m, 4H), 7.18-7.05 (m, 10H), 6.97-6.92 (m, 8H), 6.90-6.84 (m, 4H), 6.62 (dd, J=4.1, 2.5 Hz, 2H), 3.80 (s, 12H).

¹³C-NMR (151 MHz, Acetone-d₆, δ) 180.74, 157.64, 150.15, 142.11, 141.02, 130.87, 128.16, 127.34, 123.17, 119.94, 115.77, 108.97, 55.76.

### Synthesis of Diketo Precursor 7c

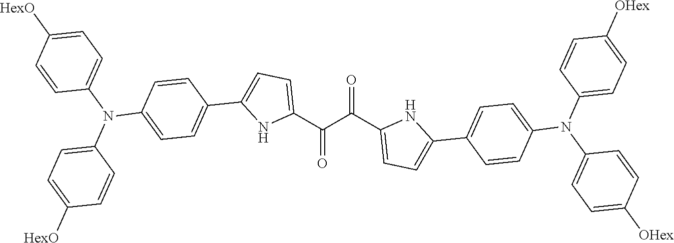

In a dry round bottomed Schlenk flask, added 10 mL of dry dichloromethane under nitrogen. Then added oxalyl chloride (0.084 mL, 0.9848 mmol, 1.0 equiv.) and the reaction mixture was cooled to −78° C. To this mixture dry pyridine was added (0.16 mL, 1.97 mmol, 2.0 equiv.) and further stirred for 40 min. at the same temperature. Then cannula transferred the solution of 1-bis(N,N-4-hexyloxydiphenyl)-4-1H-pyrrole benzene 4c (1.06 g, 1.97 mmol, 2.0 equiv.) in 10 mL of dry dichloromethane. The reaction mixture was further stirred for 15 minutes at the same temperature. The mixture was allowed to warm to RT. The reaction mixture was quenched by adding water. Organic layer was separated. Aqueous layer was extracted with dichlormethane (3*20 mL). Combined organic layer wad washed with brine solution and dried with anhydrous sodium sulfate. The crude mixture was concentrated under reduced pressure in rotary evaporator and purified by basic alumina column chromatography to isolate 465 mg of the product (Isolated yield=46%).

¹H-NMR (600 MHz, Acetone-d₆, δ) 11.09 (s, 2H), 7.85-7.66 (m, 4H), 7.20-7.02 (m, 6.96-6.92 (m, 8H), 6.89-6.85 (m, 4H), 6.63 (dd, J=4.0, 2.5 Hz, 2H), 4.00 (t, J=6.5 Hz, 8H), 1.80-1.75 (m, 8H), 1.52-1.47 (m, 8H), 1.38-1.33 (m, 16H), 0.93-0.89 (m, 12H).

### Synthesis of BF2 Bridged Product 8a

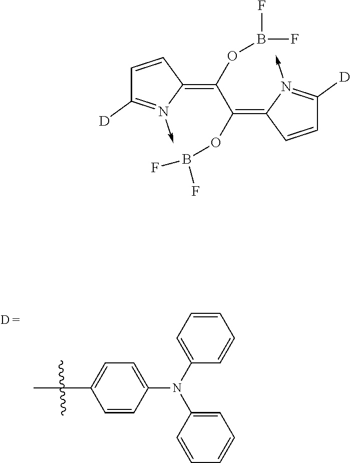

Diketo precursor 5a (0.1 g, 0.1482 mmol, 1 equiv.) taken in a pressure flask. Added dry 2 mL of THF under nitrogen followed by the addition of 2,6-di-tert-butylpyridine (1.5 mL, 6.8 mmol, 46 equiv.) and boron trifluoride etherate (1.1 mL, 8.9 mmol, 60 equiv.). The reaction mixture was stirred at 100° C. in pressure flask for 48 h. After the reaction solvent was evaporated. The crude mixture was further dissolved in minimal amount of dichloromethane. The product was precipitated dichloromethane solution by adding methanol to isolate 74 mg of the product (Yield=64%).

¹⁹F-NMR (470 MHz, Chloroform-d, δ) −139.73.

¹¹B-NMR (160 MHz, Chloroform-d, δ) 0.47.

### Synthesis of BF₂ Bridged Product 8b

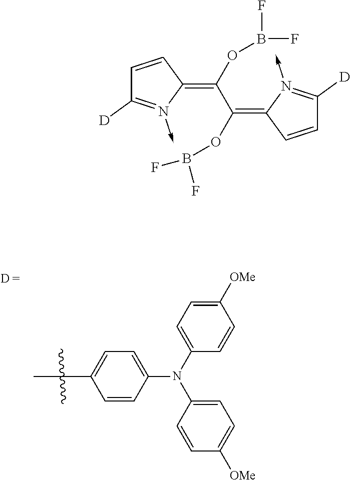

Diketo precursor 5b (0.32 g, 0.4025 mmol, 1 equiv.) taken in a pressure flask. Added dry 12 mL of THF under nitrogen followed by the addition of 2,6-di-tert-butylpyridine (5.4 mL, 24.15 mmol, 60 equiv.) and boron trifluoride etherate (2.37 mL, 24.15 mmol, 60 equiv.). The reaction mixture was stirred at 100° C. in pressure flask for 48 h. After the reaction solvent was evaporated. The crude mixture was further dissolved in minimal amount of dichloromethane. The product was precipitated dichloromethane solution by adding methanol to isolate 281 mg of the product (Yield=78%).

¹H NMR-(500 MHz, Chloroform-d, δ) 7.93 (s, 4H), 7.59 (d, J=4.2 Hz, 2H), 7.12 (d, J=24.6 Hz, 10H), 6.92 (d, J=8.4 Hz, 12H), 3.83 (s, 12H).

¹⁹F-NMR (470 MHz, Chloroform-d, δ) −153.98.

^{B-NMR (}160 MHz, Chloroform-d, δ) 4.11.

### Synthesis of BF₂ Bridged Product 8c

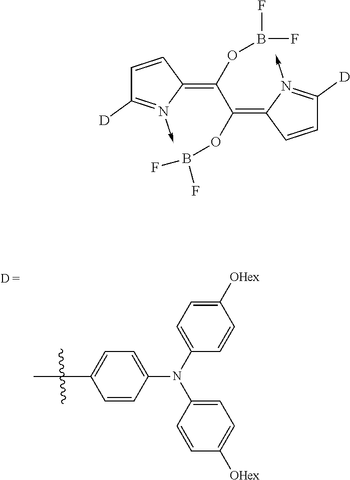

Diketo precursor 5c (0.411 g, 0.4114 mmol, 1 equiv.) taken in a pressure flask. Added dry 2 mL of THF under nitrogen followed by the addition of 2,6-di-tert-butylpyridine (4.09 mL, 18.9 mmol, 46 equiv.) and boron trifluoride etherate (3.04 mL, 24.6 mmol, 60 equiv.). The reaction mixture was stirred at 100° C. in pressure flask for 48 h. The reaction solvent was evaporated. The crude mixture was further dissolved in minimal amount of dichloromethane. The product was precipitated dichloromethane solution by adding methanol to isolate 200 mg of the product. (Yield 39%).

¹H-NMR (600 MHz, Chloroform-d, δ) 7.99-7.77 (m, 4H), 7.27-7.10 (m, 16H), 6.91-6.87 (m, 8H), 3.96 (t, J=6.5 Hz, 8H), 1.82-1.75 (m, 8H), 1.53-1.45 (m, 8H), 1.41 (s, 4H), 1.36-1.29 (m, 14H), 1.01-0.81 (m, 12H).

¹⁹F NMR (564 MHz, Acetone-d₆, δ) −151.95.

It is understood that the various embodiments described herein are by way of example only, and are not intended to limit the scope of the disclosure. For example, many of the materials and structures described herein may be substituted with other materials and structures without deviating from the spirit of the disclosure. The present compounds as disclosed may therefore include variations from the particular examples and preferred embodiments described herein, as will be apparent to one of skill in the art. It is understood that various theories as to why the embodiments work are not intended to be limiting.

We claim:

1. A compound of Formula I: 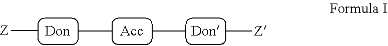
wherein Don and Don′ are each independently represented by Formula A or Formula B: 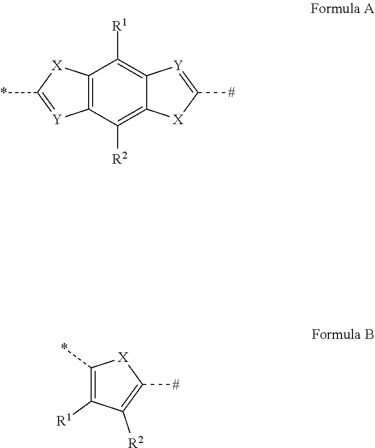
wherein, in Formula A and Formula B:
* represents the bond to Acc;
# represents the bond to Z or Z′;
each X is independently selected from the group consisting of O, S, Se, Te, GeRR′, CRR′, SiRR′, and NR.
each Y is independently selected from the group consisting of N and CR′.
R¹, R², R, R′, and R″ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and any two adjacent substituents optionally join to form a ring;
Acc is a divalent electron accepting group;
Z and Z′ are each together donating groups D and D′ respectively or accepting groups A and A′ respectively;
wherein each of Acc, Z, and Z′ may be further substituted with one or more substituents selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein any two adjacent substituents optionally join to form a ring.

2. The compound of claim 1, wherein, in Formula I:
Acc is an electron accepting group selected from the group consisting of SO₂, CF₂, imine, ketone, polycyclic aromatic, polycyclic heteroaromatic, alkyl-borate, aryl-borate, alkoxy-borate, and combinations thereof;
D and D′ are independently selected from the group consisting of alkoxy, aryloxy, heteroaryloxy, alkenyloxy, alkyloxy, allyloxy, amino, dialkylamino, diarylamino, thioalkyl, thioaryl, thioheteroaryl, and combinations thereof; and
A and A′ are independently selected from the group consisting of halogen, NO₂, CN, SO₂R, CF₃, imine, ketone, aldehyde, polycyclic aromatic, polycyclic heteroaromatic, alkyl-borate, aryl-borate, alkoxy-borate, and combinations thereof; and
wherein each of Acc, Z, and Z′ may be further substituted with one or more substituents selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
wherein any two adjacent substituents optionally join to form a ring.

3. The compound of claim 1, wherein the compound is represented by Formula II: 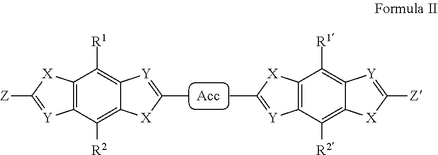
wherein, in Formula II,
Acc is a divalent electron accepting group selected from the group consisting of polycyclic aromatic, polycyclic heteroaromatic, aryl-borate, alkoxy-borate, and combinations thereof;
each X is independently selected from the group consisting of O, S, Se, Te, GeRR′, CRR′, SiRR′, and NR;
each Y is independently selected from the group consisting of N and CR″.
Z and Z′ are each together donating groups D and D′ respectively or accepting groups A and A′ respectively;
D and D′ are independently selected from the group consisting of alkoxy, aryloxy, heteroaryloxy, alkenyloxy, alkyloxy, allyloxy, amino, dialkylamino, diarylamino, thioalkyl, thioaryl, thioheteroaryl, and combinations thereof;
A and A′ are independently selected from the group consisting of halogen, NO₂, CN, SO₂R, CF₃, imine, ketone, aldehyde, polycyclic aromatic, polycyclic heteroaromatic, alkyl-borate, aryl-borate, alkoxy-borate, and combinations thereof;
R¹, R^{1′}, R², R^{2′}, R, R′, and R″ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
any two adjacent substituents optionally join to form a ring.

4. The compound of claim 1, wherein at least one of R¹ and R² is represented by one of the following structures:

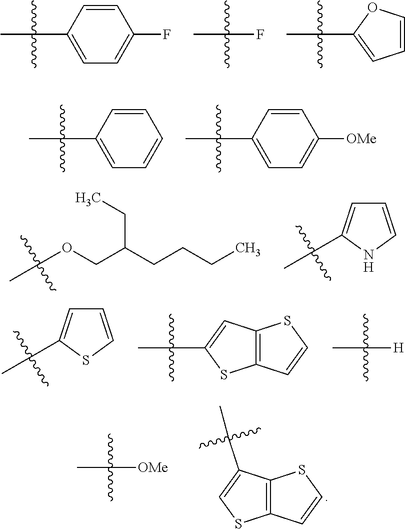

5. The compound of claim 1, wherein Acc is represented by one of the following structures: 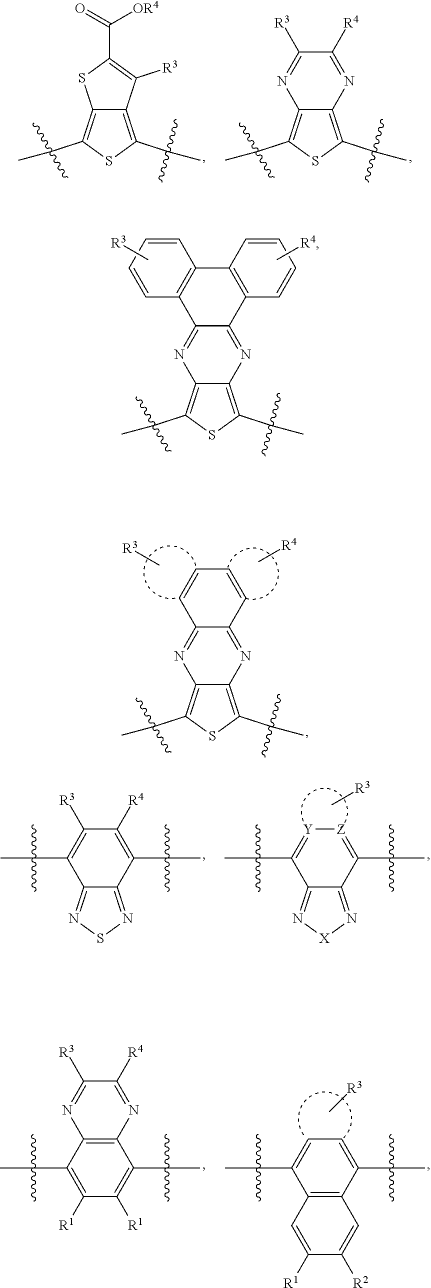 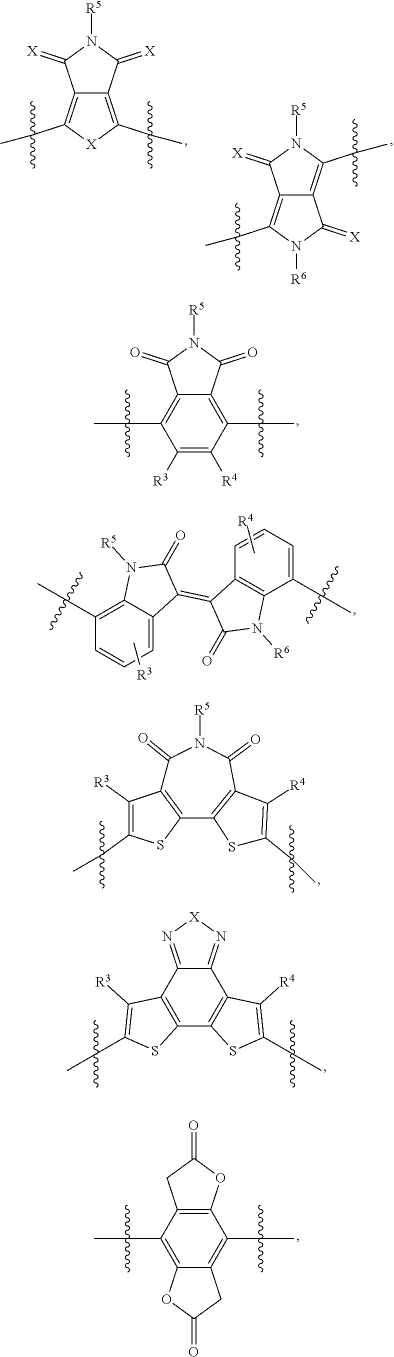 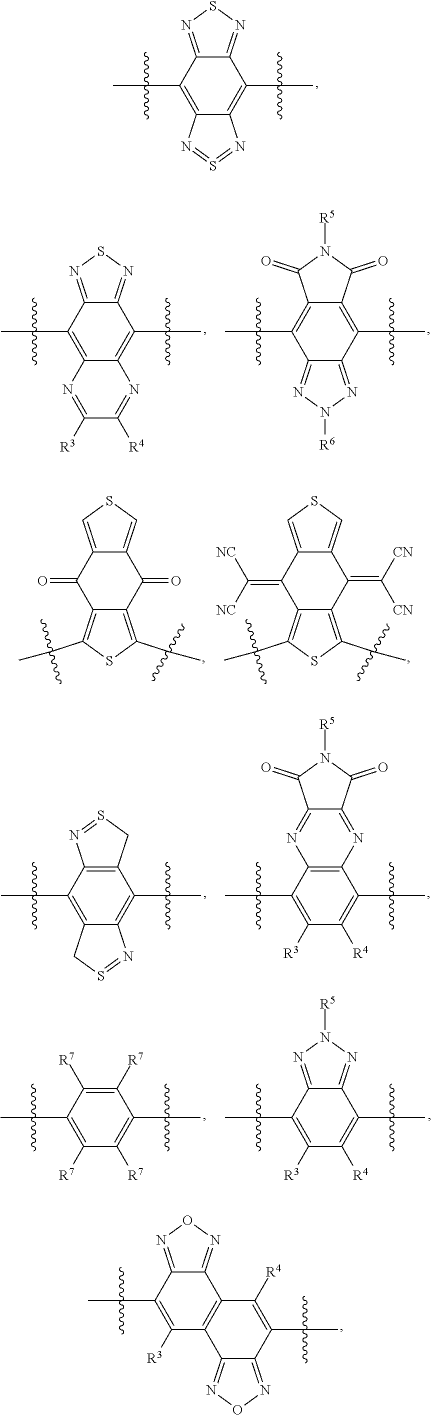 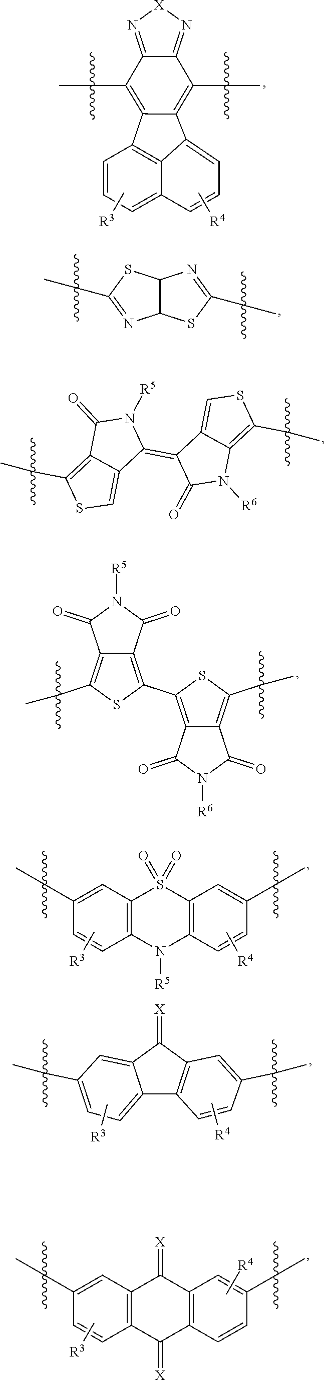 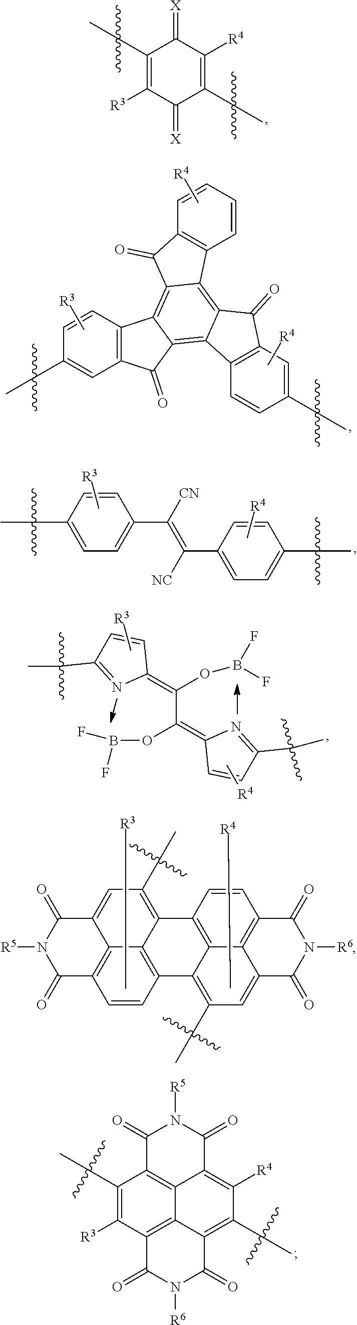
wherein wavy lines indicate bonds to Don and Don′;
wherein each X independently represents O, S, Se, NR⁵, or C(CN)₂;
wherein Y and Z independently represent CR⁴ and N;
wherein [symbol] represents optional aryl, heteroaryl, polyaromatic, or polyheteroaryl ring fusions;
wherein R³, R⁴, R⁵, and R⁶ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein each R⁷ is independently an electron-withdrawing group selected from the group consisting of halogen, haloalkyl, aryl, heteroaryl, nitrile, and combinations thereof; and
wherein any two adjacent substituents optionally join to form a ring.

6. The compound of claim 1, wherein Z and Z′ are independently represented by one of the following structures: 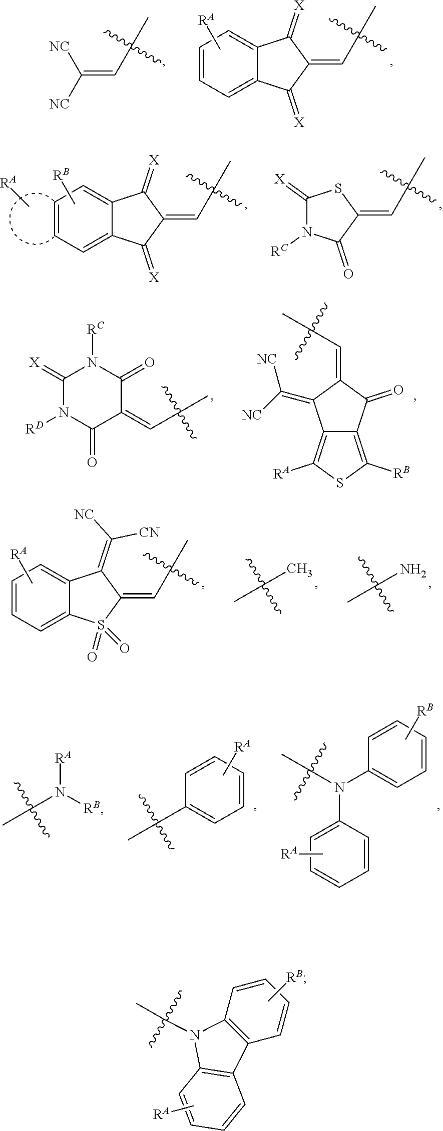
wherein each X independently represents O, S, Se, NR^{C}, or C(CN)₂;
R^{A}, R^{B}, R^{C}, and R^{D} independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein any two adjacent substituents optionally join to form a ring.

7. The compound of claim 6, wherein Z and Z′ are each an acceptor group selected from the group consisting of:

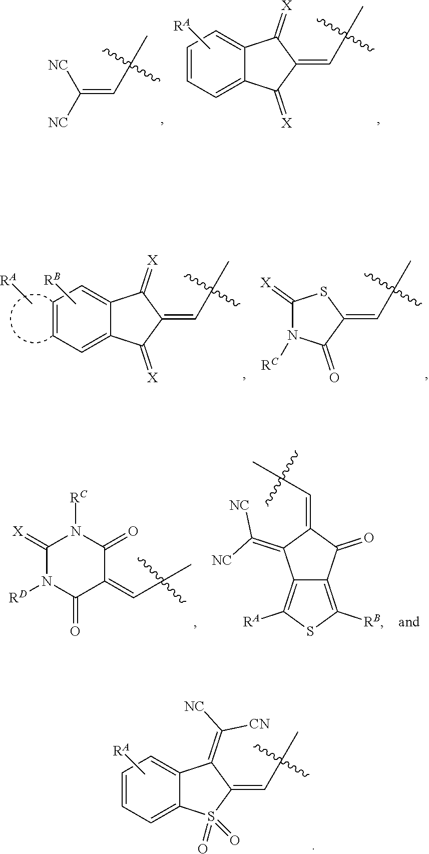

8. The compound of claim 6, wherein Z and Z′ are each a donor group selected from the group consisting of:

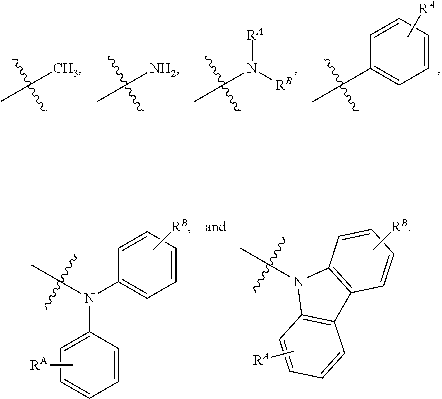

9. The compound of claim 5, wherein Acc is selected from the group consisting of:

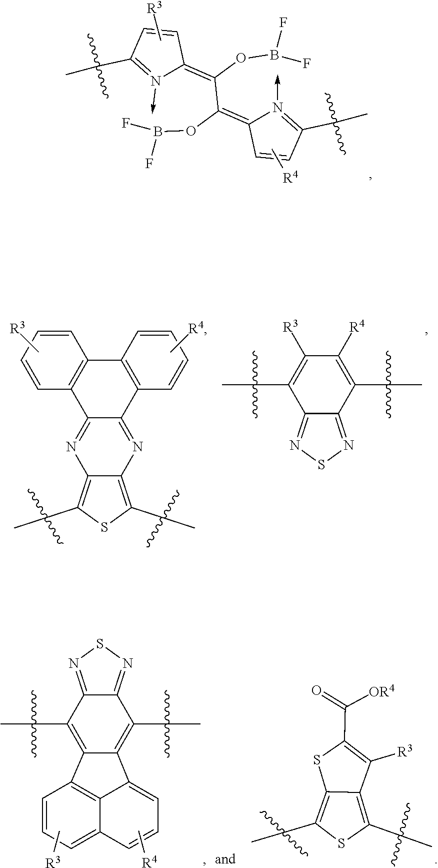

10. A compound represented by one of the following structures: 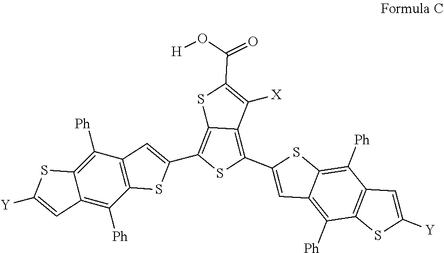
wherein, in Formula C:
X is F, CF₃, CN, SO₃H, or SO₂Me; and
Y is NH₂, NMe₂, NPh₂, N(4-OMePh)₂, N(3,4,5-(OMe)Ph)₂, or N(2,4,6-(OMe)Ph)₂;

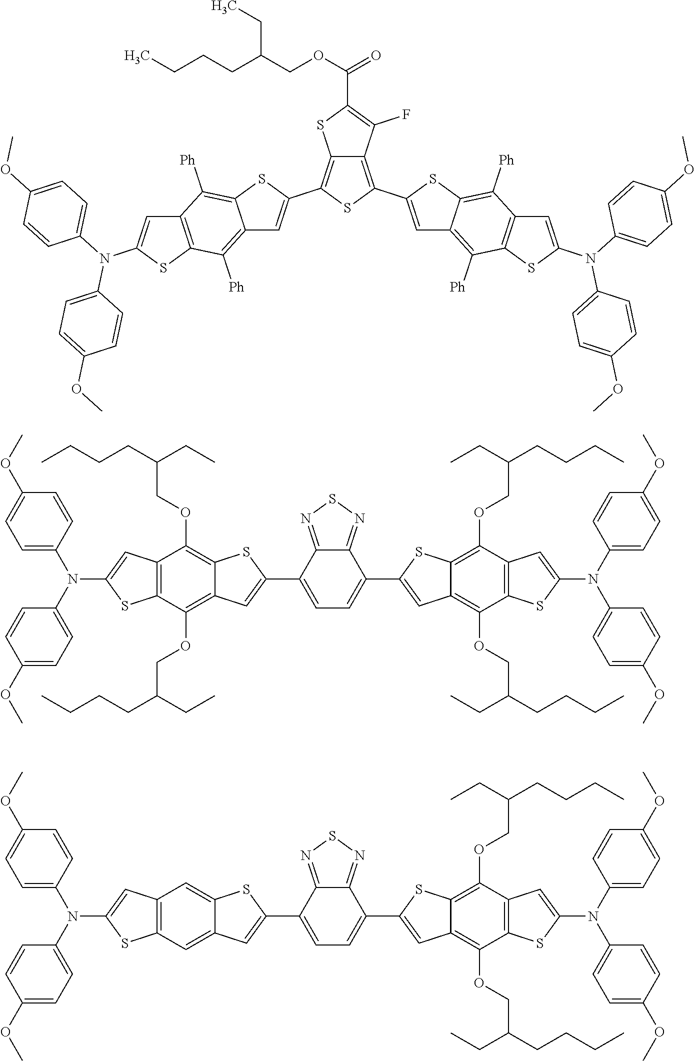

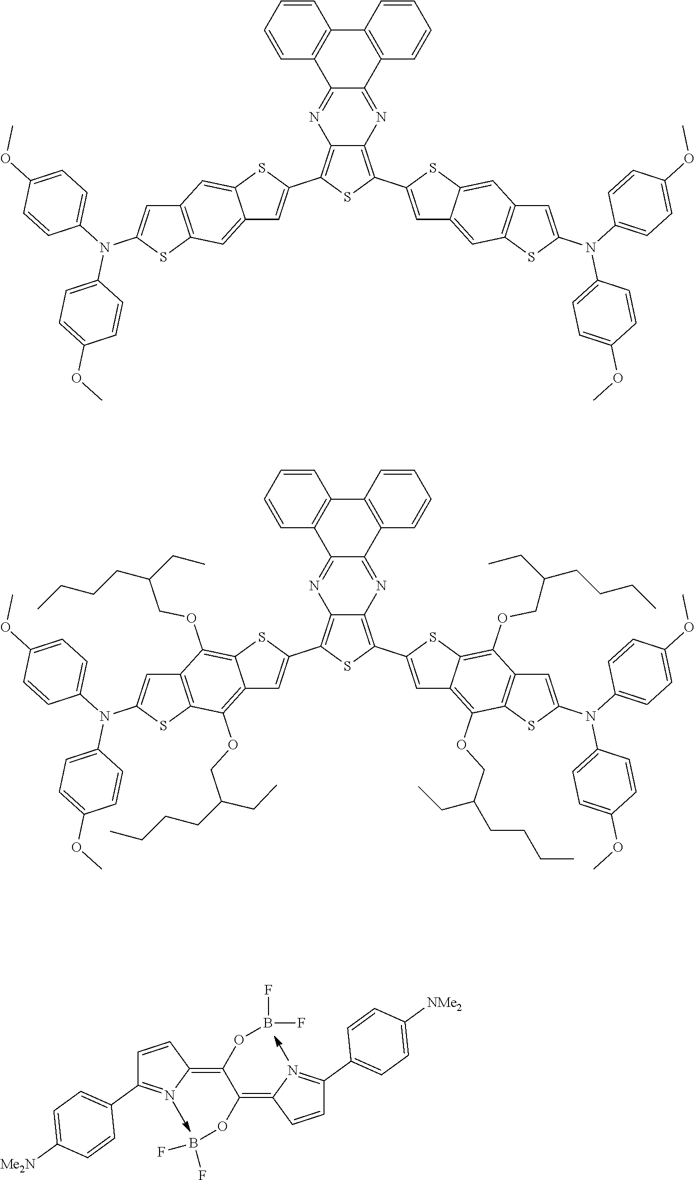 [SMILES: COc1ccc(N(c2ccc(OC)cc2)c2cc3cc4sc(-c5sc(-c6cc7cc8sc(N(c9ccc(OC)cc9)c9ccc(OC)cc9)cc8cc7s6)c6nc7c8ccccc8c8ccccc8c7nc56)cc4cc3s2)cc1 ; CCCCC(CC)COc1c2cc(N(c3ccc(OC)cc3)c3ccc(OC)cc3)sc2c(OCC(CC)CCCC)c2cc(-c3sc(-c4cc5c(OCC(CC)CCCC)c6sc(N(c7ccc(OC)cc7)c7ccc(OC)cc7)cc6c(OCC(CC)CCCC)c5s4)c4nc5c6ccccc6c6ccccc6c5nc34)sc12 ; CN(C)c1ccc(C2=N/C(=C(OB(F)F)\C(OB(F)F)=C3\C=CC(c4ccc(N(C)C)cc4)=N3)C=C2)cc1]

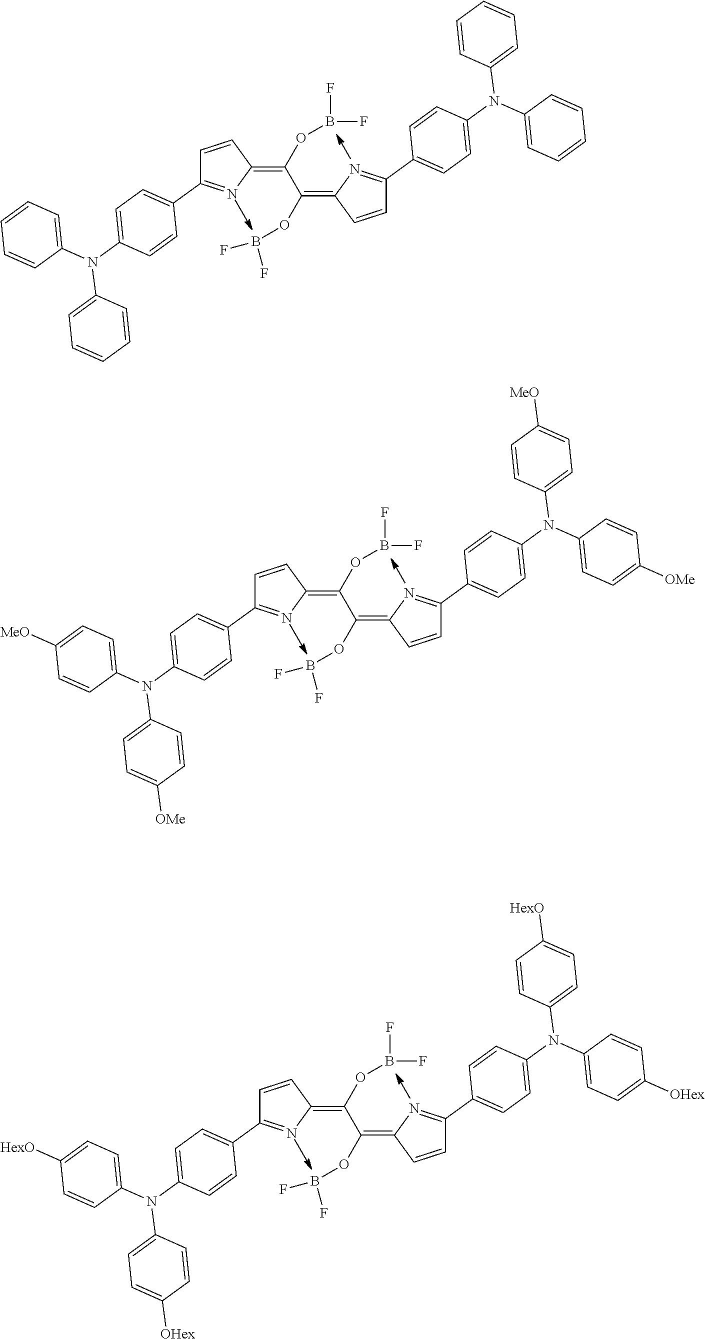

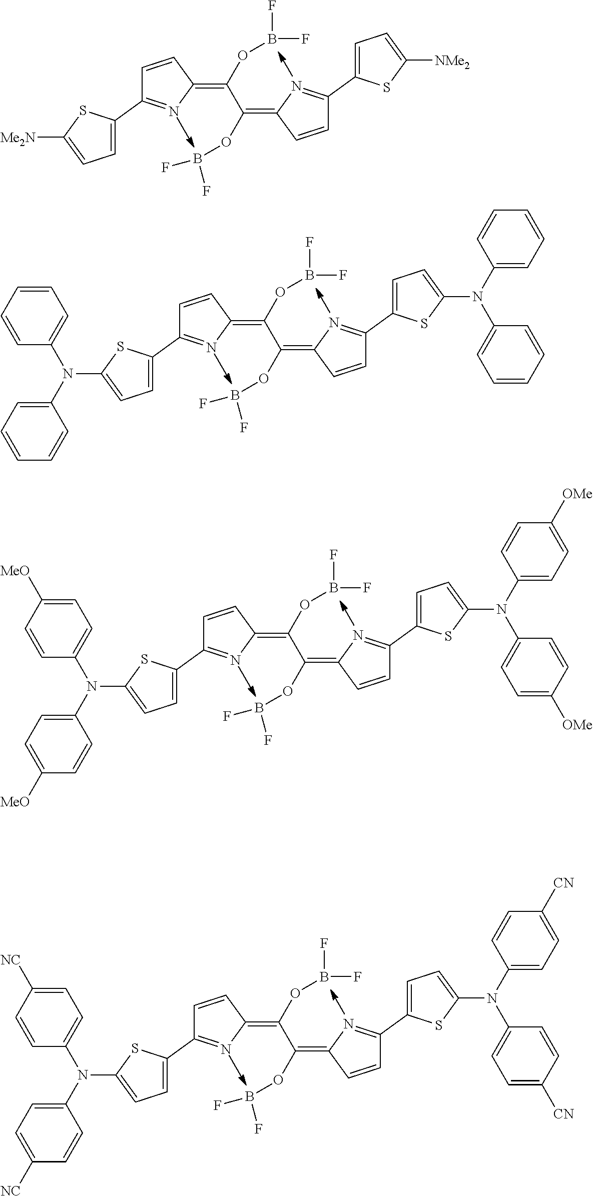 [SMILES: CN(C)c1ccc(C2=N/C(=C(OB(F)F)\C(OB(F)F)=C3\C=CC(c4ccc(N(C)C)s4)=N3)C=C2)s1 ; FB(F)OC(=C1\C=CC(c2ccc(N(c3ccccc3)c3ccccc3)s2)=N1)/C(OB(F)F)=C1/C=CC(c2ccc(N(c3ccccc3)c3ccccc3)s2)=N1 ; COc1ccc(N(c2ccc(OC)cc2)c2ccc(C3=N/C(=C(OB(F)F)\C(OB(F)F)=C4\C=CC(c5ccc(N(c6ccc(OC)cc6)c6ccc(OC)cc6)s5)=N4)C=C3)s2)cc1 ; N#Cc1ccc(N(c2ccc(C#N)cc2)c2ccc(C3=N/C(=C(OB(F)F)\C(OB(F)F)=C4\C=CC(c5ccc(N(c6ccc(C#N)cc6)c6ccc(C#N)cc6)s5)=N4)C=C3)s2)cc1]

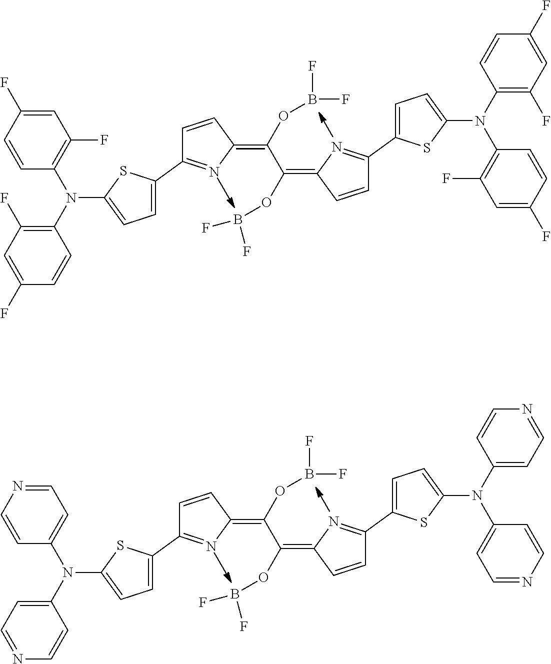 [SMILES: FB(F)OC(=C1\C=CC(c2ccc(N(c3ccc(F)cc3F)c3ccc(F)cc3F)s2)=N1)/C(OB(F)F)=C1/C=CC(c2ccc(N(c3ccc(F)cc3F)c3ccc(F)cc3F)s2)=N1 ; FB(F)OC(=C1\C=CC(c2ccc(N(c3ccncc3)c3ccncc3)s2)=N1)/C(OB(F)F)=C1/C=CC(c2ccc(N(c3ccncc3)c3ccncc3)s2)=N1]

11. An optoelectronic device comprising a compound of Formula I: 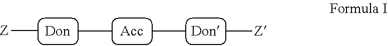
wherein Don and Don′ are each independently represented by Formula A or Formula B: 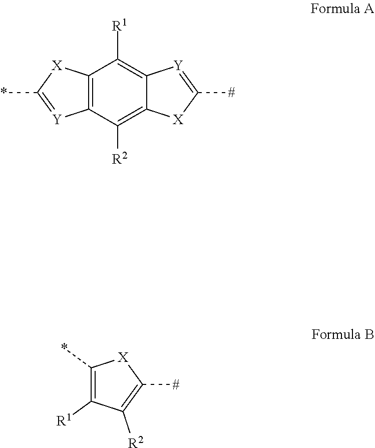
wherein, in Formula A and Formula B:
* represents the bond to Acc;
# represents the bond to Z or Z′;
each X is independently selected from the group consisting of O, S, Se, Te, GeRR′, CRR′, SiRR′, and NR.
each Y is independently selected from the group consisting of N and CR″.
R¹, R², R, R′, and R″ independently represent hydrogen or a substituent selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and any two adjacent substituents optionally join to form a ring;
Acc is a divalent electron accepting group;
Z and Z′ are each together donating groups D and D′ respectively or accepting groups A and A′ respectively;
wherein each of Acc, Z, and Z′ may be further substituted with one or more substituents selected from the group consisting of deuterium, halogen, alkyl, cycloalkyl, heteroalkyl, heterocycloalkyl, arylalkyl, alkoxy, aryloxy, amino, thioalkyl, silyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein any two adjacent substituents optionally join to form a ring.

12. The optoelectronic device of claim 11, wherein the optoelectronic device is selected from the group consisting of an organic light-emitting device (OLED), organic phototransistor, organic photovoltaic cell, and organic photodetector.

13. The optoelectronic device of claim 11, wherein the optoelectronic device is a photovoltaic cell.

14. A consumer product comprising the optoelectronic device of claim 11.

15. A formulation comprising the compound of claim 1.
